SEQUENCE LISTING (61 sequences)

SEQ ID NO: 1 - protein, 473 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGGGGGGGGGGGGGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLKSDGSFFLYSDLTVDKSRWQQGNVFSCSVMHEALHNHYTQKSLSLSPG

SEQ ID NO: 2 - protein, 474 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGGGGGGGGGGGGGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLKSDGSFFLYSDLTVDKSRWQQGNVFSCSVMHEALHNHYTQKSLSLSPGK

SEQ ID NO: 3 - protein, 226 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVDGFYPSDIAVEWESNGQPENNYKTTPPVLDSDGSFFLVSKLTVDKSRWQQGNVFSCSVMHEALHNHYTQKSLSLSPG

SEQ ID NO: 4 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVDGFYPSDIAVEWESNGQPENNYKTTPPVLDSDGSFFLVSKLTVDKSRWQQGNVFSCSVMHEALHNHYTQKSLSLSPGK

SEQ ID NO: 5 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 226 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 7 - protein, 466 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGSGGGSGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 465 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGSGGGSGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 9 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 226 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 11 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 12 - protein, 226 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 13 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVEGFYPSDIAVEWESNGQPENNYKTTPPVLDSDGSFFLVSKLTVDKSRWQQGNVFSCSVMHEALHNHYTQKSLSLSPGK

SEQ ID NO: 14 - protein, 226 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVEGFYPSDIAVEWESNGQPENNYKTTPPVLDSDGSFFLVSKLTVDKSRWQQGNVFSCSVMHEALHNHYTQKSLSLSPG

SEQ ID NO: 15 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLKSDGSFFLYSDLTVDKSRWQQGNVFSCSVMHEALHNHYTQKSLSLSPGK

SEQ ID NO: 16 - protein, 226 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLKSDGSFFLYSDLTVDKSRWQQGNVFSCSVMHEALHNHYTQKSLSLSPG

SEQ ID NO: 17 - protein, 474 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKSGGGSGGGSGGGSGGGSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLKSDGSFFLYSDLTVDKSRWQQGNVFSCSVMHEALHNHYTQKSLSLSPGK

SEQ ID NO: 18 - protein, 473 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKSGGGSGGGSGGGSGGGSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLKSDGSFFLYSDLTVDKSRWQQGNVFSCSVMHEALHNHYTQKSLSLSPG

SEQ ID NO: 19 - protein, 474 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPGKSGGGSGGGSGGGSGGGSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLKSDGSFFLYSDLTVDKSRWQQGNVFSCSVMHEALHNHYTQKSLSLSPGK

SEQ ID NO: 20 - protein, 473 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPGKSGGGSGGGSGGGSGGGSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLKSDGSFFLYSDLTVDKSRWQQGNVFSCSVMHEALHNHYTQKSLSLSPG

SEQ ID NO: 21 - protein, 474 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKSGGGSGGGSGGGSGGGSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 473 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKSGGGSGGGSGGGSGGGSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 23 - protein, 226 aa, Homo sapiens; identical to SEQ ID NO: 3

SEQ ID NO: 24 - protein, 473 aa, Homo sapiens; identical to SEQ ID NO: 1

SEQ ID NO: 25 - protein, 473 aa, Homo sapiens
DKTHTCPPCPAPELLGGPSVFLFPPKPKDTLMASRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGGGGGGGGGGGGGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLKSDGSFFLYSDLTVDKSRWQQGNVFSCSVMHEALHNHYTQKSLSLSPG

SEQ ID NO: 26 - protein, 226 aa, Homo sapiens
DKTHTCPPCPAPELLGGPSVFLFPPKPKDTLMASRTPEVTCVVVDVSHEDPEVKFNWYVDGVEVHNAKTKPPEEQYNSTYRVVSVLTVLHQDWLNGKEYKCKVSNKALPAPIEKTISKAKGQPREPQVCTLPPSRDELTKNQVSLSCAVDGFYPSDIAVEWESNGQPENNYKTTPPVLDSDGSFFLVSKLTVDKSRWQQGNVFSCSVMHEALHNHYTQKSLSLSPG

SEQ ID NO: 27 - protein, 226 aa, Homo sapiens
DKTHTCPPCPAPELLGGPSVFLFPPKPKDTLMASRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVDGFYPSDIAVEWESNGQPENNYKTTPPVLDSDGSFFLVSKLTVDKSRWQQGNVFSCSVMHEALHNHYTQKSLSLSPG

SEQ ID NO: 28 - protein, 226 aa, Homo sapiens
DKTHTCPPCPAPELLGGPSVFLFPPKPKDTLMISRTPEVTCVVVDVSHEDPEVKFNWYVDGVEVHNAKTKPPEEQYNSTYRVVSVLTVLHQDWLNGKEYKCKVSNKALPAPIEKTISKAKGQPREPQVCTLPPSRDELTKNQVSLSCAVDGFYPSDIAVEWESNGQPENNYKTTPPVLDSDGSFFLVSKLTVDKSRWQQGNVFSCSVMHEALHNHYTQKSLSLSPG

SEQ ID NO: 29 - protein, 473 aa, Homo sapiens
DKTHTCPPCPAPELLGGPSVFLFPPKPKDTLMISRTPEVTCVVVDVSHEDPEVKFNWYVDGVEVHNAKTKPPEEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGGGGGGGGGGGGGGGGDKTHTCPPCPAPELLGGPSVFLFPPKPKDTLMISRTPEVTCVVVDVSHEDPEVKFNWYVDGVEVHNAKTKPPEEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLKSDGSFFLYSDLTVDKSRWQQGNVFSCSVMHEALHNHYTQKSLSLSPG

SEQ ID NO: 30 - protein, 473 aa, Homo sapiens
DKTHTCPPCPAPELLGGPSVFLFPPKPKDTLMASRTPEVTCVVVDVSHEDPEVKFNWYVDGVEVHNAKTKPPEEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGGGGGGGGGGGGGGGGDKTHTCPPCPAPELLGGPSVFLFPPKPKDTLMISRTPEVTCVVVDVSHEDPEVKFNWYVDGVEVHNAKTKPPEEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLKSDGSFFLYSDLTVDKSRWQQGNVFSCSVMHEALHNHYTQKSLSLSPG

SEQ ID NO: 31 - protein, 473 aa, Homo sapiens
DKTHTCPPCPAPELLGGPSVFLFPPKPKDTLMASRTPEVTCVVVDVSHEDPEVKFNWYVDGVEVHNAKTKPPEEQYNSTYRVVSVLTVLHQDWLNGKEYKCKVSNKALPAPIEKTISKAKGQPREPQVYTLPPCRDKLTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGGGGGGGGGGGGGGGGGDKTHTCPPCPAPELLGGPSVFLFPPKPKDTLMASRTPEVTCVVVDVSHEDPEVKFNWYVDGVEVHNAKTKPPEEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLKSDGSFFLYSDLTVDKSRWQQGNVFSCSVMHEALHNHYTQKSLSLSPG

SEQ ID NO: 32 - protein, 250 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKPTLYNVSLVMSDTAGTCY

SEQ ID NO: 33 - protein, 250 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVCHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKPTLYNVSLVMSDTAGTCY

SEQ ID NO: 34 - protein, 4 aa, Artificial Sequence
  Linker
GGSG

SEQ ID NO: 35 - protein, 4 aa, Artificial Sequence
  Linker
SGGG

SEQ ID NO: 36 - protein, 4 aa, Artificial Sequence
  Linker
GSGS

SEQ ID NO: 37 - protein, 4 aa, Artificial Sequence
  Linker
GGGG

SEQ ID NO: 38 - protein, 5 aa, Artificial Sequence
  Linker
GGGGS

SEQ ID NO: 39 - protein, 5 aa, Artificial Sequence
  Linker
GGGGG

SEQ ID NO: 40 - protein, 6 aa, Artificial Sequence
  Linker
GSGSGS

SEQ ID NO: 41 - protein, 6 aa, Artificial Sequence
  Linker
GGSGGS

SEQ ID NO: 42 - protein, 8 aa, Artificial Sequence
  Linker
GSGSGSGS

SEQ ID NO: 43 - protein, 8 aa, Artificial Sequence
  Linker
GGSGGGSG

SEQ ID NO: 44 - protein, 8 aa, Artificial Sequence
  Linker
GGGGGGGG

SEQ ID NO: 45 - protein, 9 aa, Artificial Sequence
  Linker
GGSGGSGGS

SEQ ID NO: 46 - protein, 10 aa, Artificial Sequence
  Linker
GSGSGSGSGS

SEQ ID NO: 47 - protein, 10 aa, Artificial Sequence
  Linker
GGGGSGGGGS

SEQ ID NO: 48 - protein, 10 aa, Artificial Sequence
  Linker
GGGGGGGGGG

SEQ ID NO: 49 - protein, 12 aa, Artificial Sequence
  Linker
GSGSGSGSGSGS

SEQ ID NO: 50 - protein, 12 aa, Artificial Sequence
  Linker
GGSGGSGGSGGS

SEQ ID NO: 51 - protein, 12 aa, Artificial Sequence
  Linker
GGSGGGSGGGSG

SEQ ID NO: 52 - protein, 12 aa, Artificial Sequence
  Linker
GGGSGGGSGGGS

SEQ ID NO: 53 - protein, 12 aa, Artificial Sequence
  Linker
GGGGGGGGGGGG

SEQ ID NO: 54 - protein, 15 aa, Artificial Sequence
  Linker
GGGGSGGGGSGGGGS

SEQ ID NO: 55 - protein, 15 aa, Artificial Sequence
  Linker
GGGGGGGGGGGGGGG

SEQ ID NO: 56 - protein, 16 aa, Artificial Sequence
  Linker
GGSGGGSGGGSGGGSG

SEQ ID NO: 57 - protein, 16 aa, Artificial Sequence
  Linker
GGGGGGGGGGGGGGGG

SEQ ID NO: 58 - protein, 18 aa, Artificial Sequence
  Linker
GGSGGGSGGGSGGGSGGS

SEQ ID NO: 59 - protein, 20 aa, Artificial Sequence
  Linker
GGSGGGSGGGSGGGSGGGSG

SEQ ID NO: 60 - protein, 20 aa, Artificial Sequence
  Linker
SGGGSGGGSGGGSGGGSGGG

SEQ ID NO: 61 - protein, 20 aa, Artificial Sequence
  Linker
GGGGGGGGGGGGGGGGGGGG

(19) United States
(12) Patent Application Publication
(10) Pub. No.: US 2023/0025806 A1
(43) Pub. Date: Jan. 26, 2023

(54) STABLE COMPOSITIONS OF FC MULTIMERS

(71) Applicant: CSL BEHRING LENGNAU AG, Lengnau BE (CH)

(72) Inventors: Ahmad Mohamad ABDUL FATTAH MOHAMAD, 6th of October City (EG); Megan GARVEY, Melbourne, Victoria (AU); Michele POZZOLI, Arosio (IT); Adam TILLEY, Lucas, Victoria (AU); Peter TSIRIKIS, Malvern East, Victoria (AU)

(21) Appl. No.: 17/782,404

(22) PCT Filed: Dec. 7, 2020
(86) PCT No.: PCT/EP2020/084919

§ 371 Date: Jun. 3, 2022

(30) Foreign Application Priority Data
Dec. 6, 2019 (EP) 19214265.1

(51) Int. Cl. C07K 16/28; A61K 47/26; A61P 37/00; A61K 9/19

(52) U.S. Cl. CPC C07K 16/283; A61K 47/26; A61P 37/00; A61K 9/19

(57) ABSTRACT

The disclosure relates inter alia to an Fc multimer composition, an Fc multimer composition comprising the Fc multimer composition of the invention in lyophilized form, as well as an Fc multimer composition according to the invention for use in the treatment of an autoimmune disease or an inflammatory disease.

The invention relates inter alia to an Fc multimer composition, an Fc multimer composition comprising the Fc multimer composition of the invention in lyophilized form, as well as an Fc multimer composition according to the invention for use in the treatment of an autoimmune disease or an inflammatory disease.

### BACKGROUND OF THE INVENTION

Immunoglobulins play an important role in the immune system of mammals. They are produced by B-lymphocytes, found in blood plasma, lymph and other body secretions. The basic unit of immunoglobulins is a heterotetramer, containing 2 heavy chains and two light chains, linked by disulphide bonds. Each of these chains have a variable region at their N-terminus which form the antigen binding site, and constant regions, which are responsible for the effector functions of the immunoglobulins.

There are five major classes of immunoglobulins with differing biochemical and physiological properties: IgG (γ heavy chain), IgA (α), IgM (μ), IgD (δ) and IgE (ε). Human IgG represents the most abundant immunoglobulin in plasma, whereas IgA represents the main antibody class in external secretions such as saliva, tears and mucus of the respiratory and intestinal tracts. IgM is by far the physically largest antibody in the human circulatory system, usually being present as a pentamer of the basic immunoglobulin unit, and appears early in the course of an infection.

The Fc region (“fragment crystallizable”) is the tail region of an immunoglobulin, and comprises the two C-terminal domains of the heavy chains in the case of IgG and IgA, and the three C-terminal domains of the heavy chains in the case of IgM and IgE.

IgG products purified from human plasma, usually administered intravenously (IVIG) or subcutaneously (SCIG), are now used in a number of clinical applications. In addition to the traditional use for the treatment of primary or acquired immunodeficiencies, and infectious diseases, it has been shown that these products are also effective in the treatment of autoimmune diseases and certain neurological disorders such as CIDP.

The mechanism of action of IVIG/SCIG products in the treatment of autoimmune diseases, inflammatory and neurological disorders appears to be mainly based on the effector functions of the IgG molecule, mediated by the Fc region. The Fc region is responsible for interacting with Fc receptors and the complement system, thereby having immunomodulatory activity.

Fc receptors are receptors on the outer surface of cells of the immune system, such as human platelets, mast cells, phagocytes like macrophages and monocytes, granulocytes like neutrophils, basophils and eosinophils, as well as lymphocytes of the innate immune system (natural killer cells) or adaptive immune system (e.g., B cells). Binding of the Fc region of an antibody that via its Fab region is attached to an antigen (such as a microbial pathogen) usually leads to a connection of the pathogen to the cells of the immune system. As a result, a concerted action against the pathogen is elicited.

The complement system is a part of the immune system attacking a pathogen's cell membrane by forming transmembrane channels, which disrupt the pathogen's cell membrane. This subsequently leads to cell lysis and death of the pathogen. Further, activation of the complement system leads to stimulation of phagocytes to clear foreign and damaged material and inflammation to attract additional phagocytes.

Several strategies for replacing IVIG/SCIG for the treatment of autoimmune, inflammatory and neurological disorders are under investigation. One approach is to use only the Fc portions of the IgG in high concentration, or to assemble recombinantly several Fc portions into one molecule, for example to increase the avidity of the Fc portions to Fc receptors. For example, various configurations of multimeric Fc molecules were disclosed in WO 2008/151088 A2. Advantageous constructs with different configurations of 2 to 10 Fc portions were disclosed (see Momenta patent application WO 2015/168643 A2). The most preferred option was a trivalent human IgG1 Fc molecule, produced from two long chains of two Fc polypeptides joined by a flexible peptide linker, and two short chains of a single Fc polypeptide. Mutations such as knob-in-hole and electrostatic mutations were introduced to direct correct assembly of the construct. Another example is a hexameric Fc molecule, assembled by the addition of the C-terminal 18 amino acid tail-piece of IgM to an IgG Fc polypeptide, as disclosed e.g. in WO 2014/060712 A1 and WO 2017/129737 A1.

For therapeutic use, stable formulations of such Fc multimers are required. However, even single Fc fragments are highly susceptible to physical and chemical degradation, in particular they are highly prone to aggregation in solution. For Fc multimers, this is even more problematic. First attempts to formulate the Fc multimer in liquid form were only successful at very low protein concentration (30 mg/ml). For therapeutic use, a more concentrated solution of these proteins is highly desirable. Stable compositions with suitably high protein concentrations have not been achieved, mainly because, as mentioned above, Fc multimers tend to aggregate in solution, and the rate of aggregation increases with increasing protein concentration, leading to visible precipitates. It is known that such precipitates may be harmful when administered to a patient.

The object of the present invention is therefore to provide a stable Fc multimer composition having a higher concentration of Fc multimers, which allows convenient administration of the Fc multimer to a patient, e.g. by subcutaneous administration.

### SUMMARY OF THE INVENTION

An Fc multimer composition comprising
a) an Fc multimer at a concentration between 60 mg/ml and 180 mg/ml,
b) a pH value between 4.8 to 6.0, and
c) a stabilizer at a concentration between 200 mM and 450 mM, is provided.

The invention further relates to an Fc multimer composition, comprising the Fc multimer composition of the invention in lyophilized form.

Moreover, an Fc multimer composition of the invention for use in the treatment of an autoimmune disease or an inflammatory disease is provided.

### DETAILED DESCRIPTION OF THE INVENTION

In a first aspect, the invention relates to an Fc multimer composition comprising
a) an Fc multimer at a concentration between 60 mg/ml and 180 mg/ml,
b) a pH value between 4.8 to 6.0, and
c) a stabilizer at a concentration between 200 mM and 450 mM.

The results of the examples show that the Fc multimer composition according to the present invention is particularly stable. Especially, according to the examples, it has been found that using the conditions as given in the claims prevent protein precipitation and deamidation and lead to a decreased fragmentation rate even in Fc multimer compositions having a higher protein concentration.

The term “Fc multimer” according to the present invention describes a multimer composed of at least 2 Fc portions.

The term “Fc portion”, “Fc monomer”, “Fc fragment” or “Fc domain”, used interchangeably herein, according to the present invention describes a molecule assembled from 2 Fc polypeptides that is capable of binding an Fc receptor.

The term “Fc polypeptide” according to the present invention describes a polypeptide chain consisting of portions of the heavy chain constant domain of an antibody.

In particular, the Fc polypeptide of the present invention comprises an antibody constant domain (CH2 and/or CH3) or functional fragments thereof (e.g. fragments that are capable of (i) dimerizing with another Fc polypeptide to form an Fc portion, and (ii) binding to an Fc receptor).

Further, the Fc portion may comprise any combination of the hinge-region as well as the constant CH2 and CH3 (and CH4) domain of the immunoglobulin heavy chain constant domain, or fragments thereof.

The term “constant domain CH2/CH3/CH4” according to the present invention describes portions of the constant domain of the heavy chain of an antibody.

The term “hinge region” according to the present invention describes a flexible stretch of the polypeptide chain of the constant domain of the heavy chain of an antibody, whose usual function is the joining of the Fab and the Fc portions of an antibody, or a fragment thereof.

In particular, the Fc polypeptide of the present invention comprises or consists of a full or partial hinge region and the antibody constant domains CH2 and/or CH3 and/or CH4. More in particular, the Fc polypeptide of the present invention comprises or consists of the hinge region and the second and/or third antibody constant domain (CH2 and/or CH3), preferably the hinge region and the second and third antibody constant domains (CH2 and CH3).

An Fc polypeptide of the present invention may be derived from an IgA, IgD, IgG, IgE or IgM antibody, in particular, an Fc polypeptide of the present invention may be derived from an IgG antibody, preferably a human IgG antibody, more in particular an Fc polypeptide of the present invention may be derived from one of the IgG antibody subclasses IgG1, IgG2a, IgG2b, IgG3 or IgG4. Most preferably, the Fc polypeptide of the present invention may be derived from human IgG1.

In general, an Fc polypeptide according to the present invention does not comprise any part of an immunoglobulin that is capable of acting as an antigen-recognition region, nor any complementarity determining regions (CDRs).

The Fc polypeptide may be in a wild-type form or may comprise one or more changes in comparison to a wild-type Fc polypeptide sequence.

Such a change may be any known change of an amino acid known to the person skilled in the art, such as an amino acid substitution, an addition or a deletion. Further, a change may be conservative or any change that alters the interaction between two Fc polypeptides, between an Fc polypeptide or Fc domain and an Fc receptor, or between an Fc polypeptide or an Fc domain and the complement system.

A change may be introduced at any position of the Fc polypeptide, in particular at the hinge region, the CH2 or the CH3 domain.

Suitable changes are known to the person skilled in the art. In particular, the changes may be knob-into-hole amino acid changes, amino acid change leading to disulfide bonds, amino acid change leading to reversedly charged amino acid interactions as well as the incorporation of a peptide linker leading to an improved interaction of the Fc polypeptides in an Fc portion.

The use of engineered cavities and engineered protuberances (or the “knob-into-hole” strategy) is well known to the person skilled in the art and, e.g., described by Carter and co-workers (Ridgway et al., Protein Eng. 9:617-612, 1996; Atwell et al., J Mol Biol. 270:26-35, 1997; Merchant et al., Nat Biotechnol. 16:677-681, 1998) and is also disclosed in U.S. Pat. No. 5,731,168.

In the present invention, engineered cavities and engineered protuberances may be used in the preparation of the Fc portions described herein.

The term “engineered cavity” refers to the substitution of at least one of the original amino acid residues, e.g. in the CH3 constant domain, with a different amino acid residue having a smaller side chain volume than the original amino acid residue, thus creating a three dimensional cavity, e.g. in the CH3 constant domain.

As used herein, the term “engineered protuberance” refers to the substitution of at least one of the original amino acid residues, e.g. in the CH3 constant domain, with a different amino acid residue having a larger side chain volume than the original amino acid residue, thus creating a three dimensional protuberance, e.g. in the CH3 constant domain.

An engineered cavity may be constructed by replacing amino acids containing larger side chains such as tyrosine or tryptophan with amino acids containing smaller side chains such as alanine, valine, or threonine. Similarly, an engineered protuberance may be constructed by replacing amino acids containing smaller side chains such as alanine, valine, or threonine with amino acids containing larger side chains such as tyrosine or tryptophan. Methods for the replacement of amino acids are well known to the person skilled in the art.

In an Fc portion according to the present invention, two Fc polypeptides may interact, whereby the first Fc polypeptide may comprise an engineered cavity in its CH3 constant domain, while the second Fc polypeptide may comprise an engineered protuberance in its CH3 constant domain.

In other embodiments, an engineered cavity or an engineered protuberance in one CH3 constant domain may be created to better accommodate an original amino acid in another CH3 constant domain.

In the present invention, inter-CH3 domain disulfide bond engineering may further be applied to enhance dimer formation. For example, an amino acid, which is not cysteine, in one CH3 constant domain may be replaced by the amino acid cysteine in order to allow disulfide bond formation with the cysteine of another Fc polypeptide, specifically with the CH3 constant domain of an Fc polypeptide.

Further, the incorporation of one or more peptide linker may be used to enhance dimer formation. Such a peptide linker usually comprises flexible amino acids such as glycine and/or serine. Thereby, a first Fc polypeptide may be joined to a second Fc polypeptide by way of a peptide linker, wherein the N-terminus of the peptide linker may be joined to the C-terminus of the first Fc polypeptide through a chemical bond and the C-terminus of the peptide linker may be joined to the N-terminus of the second Fc polypeptide through a chemical bond. Suitable peptide linkers are well known to the person skilled in the art.

Moreover, two Fc polypeptides may be linked by a glycine linker, i.e. that two parts of an Fc polypeptide may be linked via an amino acid chain of two or more glycine molecules.

If an Fc polypeptide comprises one or more changes, these may be at any position of the molecule.

An Fc portion according to the present invention is usually assembled from two Fc polypeptides. In particular, the Fc portion according to the present invention may not to be understood to be a whole antibody.

An Fc portion may be composed of 2 identical Fc polypeptides. Further, an Fc portion may be composed of 2 Fc polypeptides that differ from each other.

In an Fc portion, both Fc polypeptides may be mutated as described above or both Fc polypeptides may be wild-type or one Fc polypeptide may be mutated as described above, while the other Fc polypeptide may be wild-type.

In an Fc portion, the Fc polypeptides may interact via any known kind of intermolecular and intramolecular force known to the person skilled in the art, such as covalent or non-covalent bonding. The Fc polypeptides may, for example, interact via reversedly charged amino acids, which may be introduced in their amino acid sequence by amino acid replacement as described above. Further, the Fc polypeptides may interact via disulfide bonds derived from the coupling of two thiol groups from two cysteine residues of different Fc polypeptides.

Further, the Fc polypeptides may be linked via a peptide linker as describe above.

In particular, the Fc multimer is composed of 2 to 10 Fc portions, more in particular the Fc multimer is composed of 3 to 6 Fc portions, and most in particular the Fc multimer is composed of 3 Fc portions.

In a preferred embodiment, the Fc multimer is composed of 3 to 6 Fc portions.

In another preferred embodiment, the Fc multimer is composed of 3 Fc portions.

In some embodiments, the Fc multimer comprises four polypeptides that form three Fc portions, wherein the first polypeptide comprises a first Fc polypeptide, a first linker, and a second Fc polypeptide, wherein the second polypeptide comprises a third Fc polypeptide, a second linker, and a fourth Fc polypeptide, wherein the third polypeptide comprises a fifth Fc polypeptide, wherein the fourth polypeptide comprises a sixth Fc polypeptide, wherein the first Fc polypeptide and the third Fc polypeptide form the first Fc portion, wherein the fifth Fc polypeptide and the second Fc polypeptide form the second Fc portion, and wherein the sixth Fc polypeptide and fourth Fc polypeptide form the third Fc portion.

In some embodiments, the Fc multimer does not contain an antigen-recognition region, e.g., a variable domain (e.g., V_{H}, V_{L}, a hypervariable region (HVR)) or a complementarity determining region (CDR).

In some embodiments of this aspect, each of the first and third Fc polypeptides include complementary dimerization selectivity modules that promote dimerization between the first Fc polypeptide and the third Fc polypeptide; and/or each of the second and fifth Fc polypeptides include complementary dimerization selectivity modules that promote dimerization between the second Fc polypeptide and the fifth Fc polypeptide; and/or each of the fourth and sixth Fc polypeptides include complementary dimerization selectivity modules that promote dimerization between the fourth Fc polypeptide and the sixth Fc polypeptide.

In some embodiments, the complementary dimerization selectivity modules promote selective dimerization of Fc polypeptides. In any of the Fc constructs described herein, the Fc polypeptides can have different sequences, e.g., sequences that differ by no more than 20 amino acids (e.g., no more than 15, 10 amino acids), e.g., no more than 20, 15, 10, 8, 7, 6, 5, 4, 3 or 2 amino acids, between two Fc polypeptides (i.e., between the Fc polypeptide and another Fc peptide of the Fc construct). For example, Fc polypeptide sequences of a construct described herein may be different because complementary dimerization selectivity modules of any of the Fc constructs can include an engineered cavity in the C_{H}3 antibody constant domain of one of the Fc polypeptides and an engineered protuberance in the C_{H}3 antibody constant domain of the other of the Fc polypeptides, wherein the engineered cavity and the engineered protuberance are positioned to form a protuberance-into-cavity pair of Fc polypeptides. In some embodiments, the Fc constructs include amino acid modifications in the C_{H}3 domain. In some embodiments, the Fc constructs include amino acid modifications in the C_{H}3 domain of the Fc polypeptides (one or more of the Fc polypeptides) for selective dimerization. Exemplary engineered cavities and protuberances are known in the art. In other embodiments, the complementary dimerization selectivity modules include an engineered (substituted) negatively-charged amino acid in the C_{H}3 antibody constant domain of one of the Fc polypeptides and an engineered (substituted) positively-charged amino acid in the C_{H}3 antibody constant domain of the other of the Fc polypeptides, wherein the negatively-charged amino acid and the positively-charged amino acid are positioned to promote formation of an Fc domain between complementary Fc polypeptides. Exemplary complementary amino acid changes are known in the art. In some embodiments, one or more of the Fc polypeptides are the same sequence. In some embodiments, one or more of the Fc polypeptides have the same modifications. In some embodiments, only one, two, three, or four of the Fc polypeptides have the same modifications.

In some embodiments, the Fc multimer includes at least two Fc monomers joined through a linker. In some embodiments, the Fc multimer includes at least one linker. A linker can be an amino acid spacer including 3-200 amino acids (e.g., 3-150, 3-100, 3-60, 3-50, 3-40, 3-30, 3-20, 3-10, 3-8, 3-5, 4-30, 5-30, 6-30, 8-30, 10-20, 10-30, 12-30, 14-30, 20-30, 15-25, 15-30, 18-22, and 20-30 amino acids). Suitable peptide linkers are known in the art, and include, for example, peptide linkers containing flexible amino acid residues such as glycine and serine. In certain embodiments, a linker can contain motifs, e.g., single, multiple or repeating motifs, of GS, GGS, GGSG, GGGGS, GGG, GGGG. In certain embodiments, a linker can include GS, GGS, GGSG, GGGGS, GGG, GGGG or any of SEQ ID NOs: 34-61. In some embodiments, a linker is used to connect two Fc polypeptides in a tandem series. In other embodiments, a linker is used to connect C_{L} and C_{H1} antibody constant domains. In other embodiments, a linker can also contain amino acids other than glycine and serine.

In some embodiments described herein, the Fc multimer can comprise a polypeptide comprising SEQ ID NOs: 1-31 or SEQ ID NOs: 1-31 with up to 10 (e.g., up to 9, 8, 7, 6, 5, 4, 3, 2, or 1) single amino acid modifications (e.g., substitutions, e.g., conservative substitutions). In some embodiments, the Fc polypeptides of an Fc domain of a construct can have different sequences, e.g., sequences that differ by no more than 20 amino acids (e.g., no more than 15, 10 amino acids), e.g., no more than 20, 15, 10, 8, 7, 6, 5, 4, 3 or 2 amino acids, between two Fc polypeptides (i.e., between the Fc polypeptide and another Fc polypeptide of the Fc construct).

In some embodiments, one or more polypeptides in an Fc construct contain a terminal lysine residue. In some embodiments, one or more Fc polypeptides in an Fc construct do not contain a terminal lysine residue. In some embodiments, all of the Fc polypeptides in an Fc construct contain a terminal lysine residue. In some embodiments, all of the Fc polypeptides in an Fc construct do not contain a terminal lysine residue. In some embodiments, the terminal lysine residue in an Fc polypeptide comprises, consists of, or consists essentially of the sequence of any one of SEQ ID NOs: 2, 4, 5, 7, 9, 11, 13, 15, 17, 19, and 21 may be removed to generate a corresponding Fc polypeptide that does not contain a terminal lysine residue. In some embodiments, a terminal lysine residue may be added to an Fc polypeptide comprising, consisting of, or consisting essentially of the sequence of SEQ ID NO: 1, 3, 6, 8, 10, 12, 14, 16, 18, 20, and 22-31 to generate a corresponding Fc polypeptide that contains a terminal lysine residue.

In an embodiment, the Fc multimer in the present invention is as disclosed in WO 2015/168643 A2, WO 2017/205436 A1, WO 2017/205434 A1, and WO 2018/129255 A1, hereby incorporated in their entirety by reference. Preferably, the Fc multimer comprises 2-10 Fc domains, e.g., Fc constructs having 2, 3, 4, 5, 6, 7, 8, 9, or 10 Fc domains. In some embodiments, the Fc multimer comprises 3 Fc domains.

In one aspect, the disclosure features an Fc construct that includes four polypeptides that form three Fc domains. The first polypeptide has the formula A-L-B, wherein A includes a first Fc polypeptide; L is a linker; and B includes a second Fc polypeptide. The second polypeptide has the formula A′-L′-B′, wherein A′ includes a third Fc polypeptide; L′ is a linker; and B′ includes a fourth Fc polypeptide. The third polypeptide includes a fifth Fc polypeptide, and the fourth polypeptide includes a sixth Fc polypeptide. In this aspect, A and A′ combine to form a first Fc domain, B and fifth Fc polypeptide combine to form a second Fc domain, and B′ and sixth Fc polypeptide combine to form a third Fc domain.

In some embodiments of this aspect, A and A′ each include a dimerization selectivity module that promotes dimerization between these Fc polypeptides. In other embodiments, B and the fifth Fc polypeptide each include a dimerization selectivity module that promotes dimerization between these Fc polypeptides. In yet other embodiments, B′ and the sixth Fc polypeptide each include a dimerization selectivity module that promotes dimerization between these Fc polypeptides.

In some embodiments of this aspect, one or more of A, B, A′, B′, the third polypeptide, and the fourth polypeptide consists of an Fc polypeptide. In some embodiments, each of A, B, A′, B′, the third polypeptide, and the fourth polypeptide consists of an Fc polypeptide.

In some embodiments of this aspect, each of B and B′ includes the mutations D399K and K409D, each of A and A′ includes the mutations S354C, T366W, and E357K, and each of the fifth and sixth Fc polypeptides includes the mutations Y349C, T366S, L368A, Y407V, and K370D. The numbering refers to the Kabat EU numbering system for IgG throughout this document, which is well known to the skilled person.

In some embodiments of this aspect, each of A and A′ includes the mutations D399K and K409D, each of B and B′ includes the mutations S354C, T366W, and E357K, and each of the fifth and sixth Fc polypeptides includes the mutations Y349C, T366S, L368A, Y407V, and K370D.

In some embodiments of this aspect, each of L and L′ includes at least 4, 8, 12, 14, 16, 18, or 20 glycines. In some embodiments, each of L and L′ includes 4-30, 8-30, or 12-30 glycines. In some embodiments of this aspect, each of L and L′ comprises, consists of, or consists essentially of GGGGGGGGGGGGGGGGGGGG (SEQ ID NO: 61).

In some embodiments, the Fc construct further includes a heterologous moiety, e.g., a peptide, e.g., an albumin-binding peptide joined to the N-terminus or C-terminus of B or B′, e.g., by way of a linker.

In other embodiments, the first and second polypeptides of the Fc construct have the same amino acid sequence and the third and fourth polypeptides of the Fc construct have the same amino acid sequence.

In some embodiments, the first and second polypeptide comprise, consist of, or consist essentially of the sequence of SEQ ID NO: 24, and the third and fourth polypeptide comprise, consist of, or consist essentially of the sequence of SEQ ID NO: 23. In some embodiments, the first and second polypeptide comprise, consist of, or consist essentially of the sequence of SEQ ID NO: 24, and the third and fourth polypeptide comprise, consist of or consist essentially of SEQ ID NO: 23 with a substitution at position 162 of Asp for Glu. In some embodiments, the the Fc multimer in the composition comprises two polypeptides having the amino acid sequence of SEQ ID NO: 23 and two polypeptides having the amino acid sequence of SEQ ID NO: 24. In some embodiments, the Fc multimer in the composition comprises two polypeptides having the amino acid sequence of SEQ ID NO: 23 with a substitution at position 162 of Asp for Glu, and two polypeptides having the amino acid sequence of SEQ ID NO: 24. In some embodiments, the Fc multimer in the composition comprises two polypeptides having the amino acid sequence of SEQ ID NO: 24 and two polypeptides having the amino acid sequence of SEQ ID NO: 23, where about 12% of the polypeptides have a substitution of Asp for Glu at position 162. In some embodiments of the disclosure, each of the first and second polypeptides comprises, consists of, or consists essentially of the sequence of SEQ ID NO: 24 with up to 10 (e.g., up to 9, 8, 7, 6, 5, 4, 3, 2, or 1) single amino acid modifications (e.g., substitutions, e.g., conservative substitutions), and the third and fourth polypeptide comprise, consist of, or consist essentially of the sequence of SEQ ID NO: 23 with up to 10 (e.g., up to 9, 8, 7, 6, 5, 4, 3, 2, or 1) single amino acid modifications (e.g., substitutions, e.g., conservative substitutions). In some cases, the first and second polypeptide comprise, consist of, or consist essentially of the sequence of SEQ ID NO: 29, and the third and fourth polypeptide comprise, consist of, or consist essentially of the sequence of SEQ ID NO: 28. In some embodiments of the disclosure, each of the first and second polypeptides comprises, consists of, or consists essentially of the sequence of SEQ ID NO: 29 with up to 10 (e.g., up to 9, 8, 7, 6, 5, 4, 3, 2, or 1) single amino acid modifications (e.g., substitutions, e.g., conservative substitutions), and the third and fourth polypeptide comprise, consist of, or consist essentially of the sequence of SEQ ID NO: 28 with up to 10 (e.g., up to 9, 8, 7, 6, 5, 4, 3, 2, or 1) single amino acid modifications (e.g., substitutions, e.g., conservative substitutions).

In some embodiments of this aspect of the disclosure, each of the first and third Fc polypeptides includes a complementary dimerization selectivity module that promote dimerization between the first Fc polypeptide and the third Fc polypeptide, and each of the second and fourth Fc polypeptides includes a complementary dimerization selectivity module that promote dimerization between the second Fc polypeptide and the fourth Fc polypeptide. In some embodiments, the complementary dimerization selectivity module of each of the first and second Fc polypeptides includes an engineered protuberance, and the complementary dimerization selectivity module of each of the third and fourth Fc polypeptides includes an engineered cavity.

In some embodiments, one or more of the Fc domain monomers includes an IgG hinge region or portion thereof, an IgG C_{H}2 antibody constant domain, and an IgG C_{H}3 antibody constant domain. In some embodiments, each of the Fc polypeptides includes an IgG hinge region or portion thereof, an IgG C_{H}2 antibody constant domain, and an IgG C_{H}3 antibody constant domain. In some embodiments, each of the Fc polypeptides is an IgG1 Fc polypeptide, preferably a human IgG1 Fc polypeptide.

In some embodiments of the previous two aspects of the disclosure, the N-terminal Asp in one or more of the first, second, third, and fourth polypeptides is mutated to Gln. In some embodiments, the N-terminal Asp in each of the first, second, third and fourth polypeptides is mutated to Gln.

In some embodiments, one or more of the first, second, third, and fourth polypeptides lack a C-terminal lysine. In some embodiments, each of the first, second, third, and fourth polypeptides lacks a C-terminal lysine.

In some embodiments, the first polypeptide and the second polypeptide have the same amino acid sequence and the third polypeptide and the fourth polypeptide have the same amino acid sequence. In some embodiments, the first polypeptide and the second polypeptide do not have the same amino acid sequence. In some embodiments, the third polypeptide and the fourth polypeptide do not have the same amino acid sequence.

In some embodiments, at least one of the Fc domains includes an amino acid modification that alters one or more of (i) binding affinity to one or more Fc receptors, (ii) effector functions, (iii) the level of Fc domain sulfation, (iv) half-life, (v) protease resistance, (vi) Fc domain stability, and/or (vii) susceptibility to degradation. In some embodiments, the Fc domain includes an amino acid modification that alters binding affinity to one or more Fc receptors, e.g., S267E/L328F. In some embodiments, the Fc receptor is FcγRIIb. In some cases, the modification described herein increases affinity to the FcγRIIb receptor. In some cases, the S267E/L328F modification increases binding affinity to FcγRIIb. In some embodiments, the Fc domain includes an amino acid modification that alters the level of Fc domain sulfation, e.g., 241F, 243F, 246K, 260T, or 301R. In some embodiments, the Fc domain includes an amino acid modification that alters protease resistance, e.g., selected from the following sets: 233P, 234V, 235A, and 236del; 237A, 239D, and 332E; 237D, 239D, and 332E; 237P, 239D, and 332E; 237Q, 239D, and 332E; 237S, 239D, and 332E; 239D, 268F, 324T, and 332E; 239D, 326A, and 333A; 239D and 332E; 243L, 292P, and 300L; 267E, 268F, 324T, and 332E; 267E and 332E; 268F, 324T, and 332E; 326A, 332E, and 333A; or 326A and 333A. In some embodiments, the Fc domain includes an amino acid modification that alters Fc domain susceptibility to degradation, e.g., C233X, D234X, K235X, S236X, T236X, H237X, C239X, S241X, and G249X, wherein X is any amino acid.

In some embodiments, the disclosure features an Fc construct including a) a first polypeptide including i) a first Fc polypeptide; ii) a second Fc polypeptide; and iii) a linker joining the first Fc polypeptide to the second Fc polypeptide; b) a second polypeptide including i) a third Fc polypeptide; ii) a fourth Fc polypeptide; and iii) a linker joining the third Fc polypeptide to the fourth Fc polypeptide; c) a third polypeptide includes a fifth Fc polypeptide; and d) a fourth polypeptide includes a sixth Fc polypeptide; wherein the first Fc polypeptide and fifth Fc polypeptide combine to form a first Fc domain, the second Fc polypeptide and fourth Fc polypeptide combine to form a second Fc domain, and the third Fc polypeptide and sixth Fc polypeptide combine to form a third Fc domain, and wherein at least one Fc domain includes an amino acid modification at position I253 (e.g., a single amino acid modification at position I253).

In some embodiments, the first and second polypeptides are identical to each other and the third and fourth polypeptides are identical to each other. In some embodiments, the first Fc domain includes an amino acid modification at position I253. In some cases, one or both of the first and fifth Fc polypeptides comprises an amino acid substitution at position I253. In some embodiments, the second Fc domain includes an amino acid modification at position I253. In some embodiments, one or both of the second and fourth Fc polypeptides comprises an amino acid substitution at position I253. In some embodiments, the third Fc domain includes an amino acid modification at position I253. In some embodiments, one or both of the third and sixth Fc polypeptides comprises an amino acid substitution at position I253. In some embodiments, each amino acid modification (e.g., substitution) at position I253 is independently selected from the group consisting of I253A, I253C, I253D, I253E, I253F, I253G, I253H, I253I, I253K, I253L, I253M, I253N, I253P, I253Q, I253R, I253S, I253T, I253V, I253W, and I253Y. In some embodiments, each amino acid modification (e.g., substitution) at position I253 is I253A.

In another aspect, the disclosure features an Fc construct including: a) a first polypeptide including: i). a first Fc polypeptide; ii). a second Fc polypeptide; and iii). a linker joining the first Fc polypeptide to the second Fc polypeptide; b). a second polypeptide including i). a third Fc polypeptide; ii). a fourth Fc polypeptide; and iii). a linker joining the third Fc polypeptide to the fourth Fc polypeptide; c). a third polypeptide includes a fifth Fc polypeptide; and d). a fourth polypeptide includes a sixth Fc polypeptide; wherein the first Fc polypeptide and fifth Fc polypeptide combine to form a first Fc domain, the second Fc polypeptide and fourth Fc polypeptide combine to form a second Fc domain, and the third Fc polypeptide and sixth Fc polypeptide combine to form a third Fc domain, and wherein at least one Fc domain comprises an amino acid modification at position R292 (e.g., a single amino acid modification).

In some embodiments, the first Fc domain includes an amino acid modification at position R292. In some embodiments, one or both of the first and the fifth Fc polypeptides comprises an amino acid substitution at position R292. In some embodiments, the second Fc domain includes an amino acid modification at position R292. In some embodiments, one or both of the second and the fourth Fc polypeptides comprises an amino acid substitution at position R292. In some embodiments, the third Fc domain includes an amino acid modification at position R292. In some embodiments, one or both of the third and the sixth Fc polypeptides comprises an amino acid substitution at position R292. In some embodiments, each of the first, second, and third Fc domain includes an amino acid modification (e.g., substitution) at position R292. In some embodiments, each of the first, second, and third Fc domain includes the amino acid modification (e.g., substitution) R292P (i.e., each Fc monomer has R292P modification, e.g., compared to SEQ ID NO: 23). In some embodiments, one or both of the first and fifth Fc polypeptides includes the amino acid substitution R292P, one or both of the second and fourth Fc polypeptides includes amino acid substitution R292P, and one or both of the third and sixth Fc polypeptides includes the amino acid substitution R292P.

In some embodiments, each amino acid modification (e.g., substitution) at position R292 is independently selected from R292D, R292E, R292L, R292P, R292Q, R292R, R292T, or R292Y. In some embodiments, each amino acid modification (e.g., substitution) at position R292 is R292P. In some embodiments, each of the first and third Fc domain includes the amino acid modification (e.g., substitution) I253A, and each of the first, second, and third Fc domain includes the amino acid modification (e.g., substitution) R292P. In some embodiments, one or both of the first and fifth Fc polypeptides includes the amino acid substitution I253A, one or both of the third and sixth Fc polypeptides includes the amino acid substitution I253A, one or both of the first and fifth Fc polypeptides includes the amino acid substitution R292P, one or both of the second and fourth Fc polypeptides includes amino acid substitution R292P, and one or both of the third and sixth Fc polypeptides includes the amino acid substitution R292P. In some embodiments, each of the first, second, and third Fc domain includes the amino acid modification (e.g., substitution) I253A and R292P. In some embodiments, one or both of the first and fifth Fc polypeptides includes the amino acid substitution I253A, one or both of the second and fourth Fc polypeptides includes amino acid substitution I253A, and one or both of the third and sixth Fc polypeptides includes the amino acid substitution I253A, one or both of the first and fifth Fc polypeptides includes the amino acid substitution R292P, one or both of the second and fourth Fc polypeptides includes amino acid substitution R292P, and one or both of the third and sixth Fc polypeptides includes the amino acid substitution R292P.

In some embodiments, each of the first Fc domain and third Fc domain include the amino acid substitutions I253A and R292P, and the second Fc domain includes the amino acid substitution R292P. In some cases, one or both of the first and fifth Fc polypeptides comprises the amino acid substitution I253A; one or both of the first and fifth Fc polypeptides comprises the amino acid substitution R292P; one or both of the third and sixth Fc polypeptides comprises the amino acid substitution I253A; one or both of the third and sixth Fc polypeptides comprises the amino acid substitution R292P; and one or both of the second and fourth Fc polypeptides comprises the amino acid substitution R292P.

In some embodiments, the second Fc domain includes the amino acid substitution I253A. In some embodiments, one or both of the second and fourth Fc polypeptides comprises the amino acid substitution I253A. In some embodiments, each of the first Fc domain and third Fc domain include the amino acid substitution I253A. In some embodiments, one or both of the first and fifth Fc polypeptides comprises the amino acid substitution I253A and one or both of the third and sixth Fc polypeptides comprises the amino acid substitution I253A. In some embodiments, each of the first Fc domain, second Fc domain, and third Fc domain include the amino acid substitution I253A. In some embodiments, one or both of the first and fifth Fc polypeptides comprises the amino acid substitution I253A, one or both of the second and fourth Fc polypeptides comprises the amino acid substitution I253A, and one or both of the third and sixth Fc polypeptides comprises the amino acid substitution I253A.

In some embodiments, the second Fc domain includes the amino acid substitution R292P. In some embodiments, one or both of the second and fourth Fc polypeptides comprises the amino acid substitution R292P. In some embodiments, the second Fc domain includes the amino acid substitutions I253A and R292P. In some embodiments, one or both of the second and fourth Fc polypeptides comprises the amino acid substitution I253A, and one or both of the second and fourth Fc polypeptides comprises the amino acid substitution R292P. In some embodiments, each of the first Fc domain and third Fc domain include the amino acid substitution I253A, and the second Fc domain includes the amino acid substitution R292P. In some embodiments, one or both of the first and fifth Fc polypeptides comprises the amino acid substitution I253A; one or both of the third and sixth Fc polypeptides comprises the amino acid substitution I253A; and one or both of the second and fourth Fc polypeptides comprises the amino acid substitution R292P.

In some embodiments, each of the first Fc domain and third Fc domain include the amino acid substitution I253A, and the second Fc domain includes the amino acid substitution I253A and R292P. In some embodiments, one or both of the first and fifth Fc polypeptides comprises the amino acid substitution I253A; one or both of the third and sixth Fc polypeptides comprises the amino acid substitution I253A; one or both of the second and fourth Fc polypeptides comprises the amino acid substitution I253A; and one or both of the second and fourth Fc polypeptides comprises the amino acid substitution R292P. In some embodiments, each of the first Fc domain and third Fc domain include the amino acid substitution R292P. In some embodiments, one or both of the first and fifth Fc polypeptides comprises the amino acid substitution R292P and one or both of the third and sixth Fc polypeptides comprises the amino acid substitution R292P.

In some embodiments, the first Fc domain and third Fc domain include the amino acid substitution R292P, and the second Fc domain includes the amino acid substitution I253A. In some embodiments, one or both of the first and fifth Fc polypeptides comprises the amino acid substitution R292P; one or both of the third and sixth Fc polypeptides comprises the amino acid substitution R292P; and one or both of the second and fourth Fc polypeptides comprises the amino acid substitution I253A. In some embodiments, each of the first Fc domain and third Fc domain include I253A and R292P (e.g., include the amino acid substitutions I253A and R292P). In some embodiments, one or both of the first and fifth Fc polypeptides comprises the amino acid substitution I253A; one or both of the first and fifth Fc polypeptides comprises the amino acid substitution R292P; one or both of the third and sixth Fc polypeptides comprises the amino acid substitution I253A; and one or both of the third and sixth Fc polypeptides comprises the amino acid substitution R292P.

In some embodiments, each of the first Fc domain and third Fc domain include the amino acid substitutions I253A and R292P, and the second Fc domain includes the amino acid substitution I253A. In some embodiments, one or both of the first and fifth Fc polypeptides comprises the amino acid substitution I253A; one or both of the first and fifth Fc polypeptides comprises the amino acid substitution R292P; one or both of the third and sixth Fc polypeptides comprises the amino acid substitution I253A; one or both of the third and sixth Fc polypeptides comprises the amino acid substitution R292P; and one or both of the second and fourth Fc polypeptides comprises the amino acid substitution I253A. In some embodiments, each of the first Fc domain, second Fc domain, and third Fc domain include the amino acid substitution R292P. In some embodiments, one or both of the first and fifth Fc polypeptides comprises the amino acid substitution R292P; one or both of the second and fourth Fc polypeptides comprises the amino acid substitution R292P; and one or both of the third and sixth Fc polypeptides comprises the amino acid substitution R292P.

In some embodiments, each of the first Fc domain and third Fc domain include the amino acid substitution R292P, and the second Fc domain includes the amino acid substitutions I253A and R292P. In some embodiments, one or both of the first and fifth Fc polypeptides comprises the amino acid substitution R292P; one or both of the third and sixth Fc polypeptides comprises the amino acid substitution R292P; one or both of the second and fourth Fc polypeptides comprises the amino acid substitution I253A; and one or both of the second and fourth Fc polypeptides comprises the amino acid substitution R292P. In some embodiments, each of the first Fc domain, second Fc domain, and third Fc domain include the amino acid substitutions I253A and R292P. In some embodiments, one or both of the first and fifth Fc polypeptides comprises the amino acid substitution I253A; one or both of the first and fifth Fc polypeptides comprises the amino acid substitution R292P; one or both of the second and fourth Fc polypeptides comprises the amino acid substitution I253A; one or both of the second and fourth Fc polypeptides comprises the amino acid substitution R292P; one or both of the third and sixth Fc polypeptides comprises the amino acid substitution I253A; and one or both of the third and sixth Fc polypeptides comprises the amino acid substitution R292P.

In some embodiments, each of the first, second, and third Fc domains include the amino acid substitution R292P. In some embodiments, one or both of the first and fifth Fc polypeptides comprises the amino acid substitution R292P. In some embodiments, one or both of the third and sixth Fc polypeptides comprises the amino acid substitution R292P. In some embodiments, one or both of the second and fourth Fc polypeptides comprises the amino acid substitution R292P.

In some embodiments, the Fc constructs described herein do not include an antigen-recognition region, e.g., a variable domain or a complementarity determining region (CDR). In some embodiments, the Fc construct (or an Fc domain within an Fc construct) is formed entirely or in part by association of Fc polypeptides that are present in different polypeptides. In certain embodiments, the Fc construct does not include an additional domain (e.g., an IgM tailpiece or an IgA tailpiece) that promotes association of two polypeptides. In other embodiments, covalent linkages (e.g., disulfide bridges) are present only between two Fc polypeptides that join to form an Fc domain. In other embodiments, the Fc construct does not include covalent linkages (e.g., disulfide bridges) between Fc domains. In still other embodiments, the Fc construct provides for sufficient structural flexibility such that all or substantially all of the Fc domains in the Fc construct are capable of simultaneously interacting with an Fc receptor on a cell surface. In one embodiment, the Fc polypeptides are different in primary sequence from wild-type or from each other in that they have dimerization selectivity modules.

In another aspect, the disclosure features compositions and methods for promoting selective dimerization of Fc polypeptides. The disclosure includes an Fc domain wherein the two Fc polypeptides of the Fc domain include identical mutations in at least two positions within the ring of charged residues at the interface between C_{H}3 antibody constant domains. The disclosure also includes a method of making such an Fc domain, including introducing complementary dimerization selectivity modules having identical mutations in two Fc polypeptide sequences in at least two positions within the ring of charged residues at the interface between C_{H}3 antibody constant domains. The interface between C_{H}3 antibody constant domains consists of a hydrophobic patch surrounded by a ring of charged residues. When one C_{H}3 antibody constant domain comes together with another, these charged residues pair with residues of the opposite charge. By reversing the charge of both members of two or more complementary pairs of residues, mutated Fc polypeptides remain complementary to Fc polypeptides of the same mutated sequence, but have a lower complementarity to Fc polypeptides without those mutations. In this embodiment, the identical dimerization selectivity modules promotes homodimerization. Such Fc domains include Fc polypeptides containing the double mutants K409D/D399K, K392D/D399K, E357K/K370E, D356K/K439D, K409E/D399K, K392E/D399K, E357K/K370D, or D356K/K439E. In another embodiment, an Fc domain includes Fc polypeptides including quadruple mutants combining any pair of the double mutants, e.g., K409D/D399K/E357K/K370E.

In another embodiment, in addition to the identical dimerization selectivity modules, the Fc polypeptides of the Fc domain include complementary dimerization selectivity modules having non-identical mutations that promote specific association (e.g., engineered cavity and protuberance). As a result, the two Fc polypeptides include two dimerization selectivity modules and remain complementary to each other, but have a decreased complementarity to other Fc polypeptides. This embodiment promotes heterodimerization between a cavity-containing Fc polypeptide and a protuberance-containing Fc polypeptide. In one example, the complementary dimerization selectivity modules having non-identical mutations in charged pair residues of both Fc polypeptides are combined with a protuberance on one Fc polypeptide and a cavity on the other Fc polypeptide. In another embodiment, the Fc polypeptides of the Fc domain include complementary dimerization selectivity modules having non-identical mutations that promote specific association (e.g., engineered cavity and protuberance), and do not include the identical dimerization selectivity modules.

In any of the Fc constructs described herein, it is understood that the order of the Fc polypeptides is interchangeable. For example, in a polypeptide having the formula A-L-B, the carboxy terminus of A can be joined to the amino terminus of L, which in turn is joined at its carboxy terminus to the amino terminus of B. Alternatively, the carboxy terminus of B can be joined to the amino terminus of L, which in turn is joined at its carboxy terminus to the amino terminus of C. Both of these configurations are encompassed by the formula A-L-B.

The properties of these constructs allow for the efficient generation of substantially homogenous compositions. The degree of homogeneity of a composition influences the pharmacokinetics and in vivo performance of the composition. Such homogeneity in a composition is desirable in order to ensure the safety, efficacy, uniformity, and reliability of the composition. An Fc construct of the disclosure can be in a population or composition that is substantially homogenous (e.g., at least 85%, 90%, 95%, 98%, or 99% homogeneous).

As described in further detail herein, the disclosure features substantially homogenous compositions containing Fc constructs that all have the same number of Fc domains, as well as methods of preparing such substantially homogenous compositions.

An Fc construct of the disclosure can be in a pharmaceutical composition that includes a substantially homogenous population (e.g., at least 85%, 90%, 95%, 98%, or 99% homogeneous) of the Fc construct having 2-10 Fc domains (e.g., 2-8 Fc domains, 2-6 Fc domains, 2-4 Fc domains, 2-3 Fc domains, 3-5 Fc domains, or 5-10 Fc domains) e.g., a construct having 2, 3, 4, 5, 6, 7, 8, 9, or 10 Fc domains, such as those described herein. Consequently, pharmaceutical compositions can be produced that do not have substantial aggregation or unwanted multimerization of Fc constructs.

Moreover, the Fc multimer may be composed of 6 Fc portions, wherein these Fc portions are IgG Fc portions and whereby the addition of the C-terminal 18 amino acid tail-piece of IgM leads to the hexamerization of IgG. Preferred examples of such hexamers are disclosed in WO 2017/129737 A1. For example, the Fc multimer comprises six IgG Fc domains. Each of the IgG Fc domains comprises two Fc polypeptides and each Fc polypeptide comprises an IgG Fc polypeptide and an IgM tailpiece.

In a preferred embodiment, the Fc polypeptide further comprises an IgG hinge region and the Fc polypeptide does not comprise a Fab polypeptide.

For example, in one embodiment, the Fc polypeptide comprises an IgG1 hinge region, an IgG1 Fc region, and an IgM tailpiece, and does not comprise a Fab polypeptide. In a preferred embodiment, the Fc polypeptide is SEQ ID NO: 32 and has up to 5 conservative amino acid changes.

In a preferred embodiment, the Fc polypeptide comprises an IgG1 hinge region, an IgG1 Fc region, and an IgM tailpiece, wherein the IgG1 Fc region has a cysteine instead of a leucine at position 309 (according to the EU numbering), and wherein the Fc polypeptide does not comprise a Fab polypeptide and the Fc polypeptide is SEQ ID NO: 33. In one embodiment, the Fc polypeptide is SEQ ID NO: 33 with up to 5 conservative amino acid changes.

In general, an Fc multimer may be composed of identical Fc portions. Further, an Fc multimer may be composed of Fc portions that all differ from each other or that partly are identical and partly differ from each other in any combination.

In an Fc multimer, all Fc portions may be mutated or none Fc portion may be mutated or any number of Fc portions may be mutated. In particular, the Fc portion according to the present invention may not to be understood to be a whole antibody.

The Fc polypeptide, the Fc portion or the Fc multimer may be produced by any process for antibody production, Fc portion production or protein production known to the person skilled in the art. Methods for the production of Fc polypeptides, Fc portions and Fc multimers are generally well-known to the person skilled in the art. For example, the Fc polypeptide, the Fc portion or the Fc multimer may be produced with the help of a mammalian cell, a bacterial cell or an insect cell after introducing the nucleic acid encoding the protein into said mammalian cell, bacterial cell or insect cell. Subsequently the Fc polypeptide, the Fc portion or the Fc multimer may be isolated from the mammalian cell, bacterial cell or insect cell or be recovered from the supernatant.

The Fc multimer composition according to the present invention comprises an Fc multimer at a concentration between 60 mg/ml and 180 mg/ml.

The Fc multimer composition according to the present invention in particular comprises an Fc multimer at a concentration between 70 mg/ml and 160 mg/ml, more in particular at a concentration between 80 mg/ml and 120 mg/ml, even more in particular at a concentration between 90 mg/ml and 110 mg/ml and most in particular at a concentration between 95 mg/ml and 105 mg/ml.

Further, Fc multimer composition according to the present invention in particular comprises an Fc multimer at a concentration between 70 mg/ml and 160 mg/ml, more in particular at a concentration between 70 mg/ml and 140 mg/ml, even more in particular at a concentration between 70 mg/ml and 120 mg/ml and most in particular at a concentration between 70 mg/ml and 110 mg/ml.

In a preferred embodiment, the Fc multimer composition according to the present invention comprises an Fc multimer at a concentration between 70 mg/ml and 110 mg/ml.

Further, Fc multimer composition according to the present invention in particular comprises an Fc multimer at a concentration between 90 mg/ml and 180 mg/ml, more in particular at a concentration between 110 mg/ml and 180 mg/ml, even more in particular at a concentration between 115 mg/ml and 160 mg/ml and most in particular at a concentration between 120 mg/ml and 160 mg/ml.

In another preferred embodiment, the Fc multimer composition according to the present invention comprises an Fc multimer at a concentration between 110 mg/ml and 180 mg/ml, in particular 120 mg/ml or 160 mg/ml.

The protein concentration of the Fc multimer composition according to the present invention may be measured by suitable techniques known by the person skilled in the art. For example, the protein concentration of the Fc multimer composition according to the present invention may be measured by absorption spectroscopy techniques, colorimetry (such as Bradford test, Biuret assay, bicinchoninic acid assay (BCA assay) or Lowry assay), gravimetric analysis (e.g. Kjeldahl), quantitative amino acid analysis (such as total hydrolysis, end group analysis and Edman degradation) or by refractometry. Preferably, UV spectroscopy measuring absorption at 280 nm was used for measuring the protein concentration of the Fc multimer composition of the present invention.

The Fc multimer composition according to the present invention further comprises a pH value between 4.8 and 6.0.

In particular, the Fc multimer composition according to the present invention comprises a pH value between 4.9 and 5.8, more in particular, the Fc multimer composition according to the present invention comprises a pH value between 5.0 and 5.6, and most in particular, the Fc multimer composition according to the present invention comprises a pH value between 5.0 and 5.5.

In a preferred embodiment, the Fc multimer composition according to the present invention comprises a pH value between 5.0 and 5.5.

The pH value of the Fc multimer composition of the present invention may be detected by any technique for measuring a pH value known to the person skilled in the art, such as a pH meter.

The Fc multimer composition according to the present invention further comprises a stabilizer at a concentration between 200 mM and 450 mM.

The term “stabilizer” according to the present invention may be any substance preventing degradation of any component of the composition of the present invention. In particular, the stabilizer is a substance preventing degradation and/or aggregation of the Fc multimer and/or an Fc portion of the composition of the present invention.

Suitable stabilizers are well-known to the person skilled in the art. For example, a suitable stabilizer may be any antioxidant (for e.g. preventing autooxidation), any sequestrant (for e.g. inactivating metal ions that may act as catalysts) or any ultraviolet stabilizer (for e.g. preventing degradation due to ultraviolet radiation).

In particular, the stabilizer used in the present invention may be a polyol and/or an amino acid.

The polyol used as a stabilizer in the composition of the present invention may be an organic molecule based on 2 to 20 carbon atoms, in particular 3 to 18 carbon atoms, more in particular 4 to 15 carbon atoms, most in particular 5 to 12 carbon atoms. Further, the polyol may be linear or branched. Moreover, the polyol may be a circular molecular comprising one, two or more circles. The polyol may comprise at least 2, 3, 4, 5, 6, 7, or 8 hydroxy groups. The polyol may further be aliphatic, cycloaliphatic or aromatic.

Suitable polyols used as a stabilizer in the composition of the present invention are well-known to the person skilled in the art and may be a disaccharide, glycerol, mannitol, inositol, xylitol, erythritol and/or adonitol. More in particular the stabilizer is a disaccharide, such as sucrose or trehalose, and/or sorbitol.

Suitable amino acids used as a stabilizer in the composition of the present invention are well-known to the person skilled in the art. In particular, natural amino acids, such as alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and/or valine may be used as a stabilizer in the composition of the present invention. More in particular, proline or arginine may be used as a stabilizer in the composition of the present invention.

In a preferred embodiment, the Fc multimer composition according to the present invention comprises as stabilizer a polyol, in particular a disaccharide, preferably sucrose or trehalose, or reduced saccharides such as sorbitol.

The composition according to the present invention may comprise one stabilizer or may comprise a mixture of 2, 3, 4, 5 or more stabilizers.

The composition according to the present invention may comprise from 50 mM to 500 mM stabilizer, in particular from 100 mM to 400 mM stabilizer, more in particular from 200 mM to 300 mM stabilizer, most in particular from 225 mM to 275 mM stabilizer.

The composition according to the present invention may comprise at least 200 mM sucrose, in particular from 200 mM to 1000 mM sucrose, more in particular from 200 mM to 750 mM sucrose, and most in particular from 220 mM to 500 mM sucrose. If sucrose is used in combination with one or more other stabilizers, lower concentrations may also be used, for example at least 100 mM sucrose, or from 100 mM to 250 mM sucrose.

The composition according to the present invention may comprise 100 mM to 500 mM trehalose, in particular from 150 mM to 400 mM trehalose, more in particular from 200 mM to 300 mM trehalose.

The composition according to the present invention may comprise from 50 mM to 500 mM sorbitol, in particular from 100 mM to 400 mM sorbitol, more in particular from 200 mM to 300 mM sorbitol, most in particular from 225 mM to 275 mM sorbitol.

In the Fc multimer composition according to the present invention, the molar ratio of Fc multimer to stabilizer may be between 1:100 and 1:800, in particular between 1:300 and 1:650, more in particular between 1:350 and 1:500 and most in particular between 1:400 and 1:425.

In a preferred embodiment, in the Fc multimer composition according to the present invention, the molar ratio of Fc multimer to stabilizer is between 1:300 and 1:650, in particular between 1:400 and 1:425. Preferably, the Fc multimer composition of this embodiment contains less than 10%, more preferably less than 7%, even more preferably less than 5%, most preferably less than 3.5% aggregates (determined by SEC) when stored for 12 months at 2-8° C.

Preferably, the Fc multimer composition of this embodiment contains less than 15 more preferably less than 10 even more preferably less than 8 most preferably less than 6.5 aggregates when stored at 25° C.

Moreover, the composition according to the present invention may comprise a buffer or may not comprise a buffer.

In a preferred embodiment, the Fc multimer composition according to the invention comprises a buffer.

The term “buffer” according to the present invention describes comprising a mixture of a weak acid and its conjugate base, or vice versa, which in its buffer region keeps its pH at a nearly constant value when further acids or bases are added to it.

The Fc multimer composition of the present invention may comprise one or more buffers. Suitable buffers for the present invention are well known to the person skilled in the art. In general, any buffer suitable for pharmaceutical applications, in particular suitable for being administered to a subject's body, such as a human patient, may be used. Examples of suitable buffers in the required pH range for the present invention are phosphate buffer, glutamate buffer, acetate buffer, histidine buffer and/or citrate buffer. In particular, the buffer is an acetate buffer, a histidine buffer or a citrate buffer.

In a preferred embodiment, the Fc multimer composition according to the present invention comprises a buffer, wherein the buffer is an acetate buffer, a histidine buffer or a citrate buffer. In a preferred embodiment, the Fc multimer composition according to the present invention comprises a histidine buffer.

The composition according to the present invention may further comprise an antioxidant. The term “antioxidant” according to the present invention describes a substance inhibiting oxidation. Preferably, the antioxidant inhibits oxidation of the Fc multimer or an Fc portion or Fc polypeptide of the Fc multimer of the composition. Suitable antioxidants are well known to the person skilled in the art. For example, any antioxidant suitable for pharmaceutical applications, in particular suitable for being administered to a subject's body, such as a human patient, may be used. The Fc multimer composition of the present invention may comprise one or more antioxidants.

Examples of suitable antioxidants are ascorbic acid, vitamin E, reduced glutathione (GSH) and/or methionine. In particular, the Fc multimer composition according to the present invention may comprise methionine.

Suitable amounts of antioxidant that may be used in the present invention are well known to the person skilled in the art and may also depend on the type of the antioxidant. In general, the Fc multimer composition may comprise from 0.01 mM to 100 mM antioxidant, in particular from 0.05 mM to 50 mM antioxidant, more in particular from 0.1 mM to 30 mM antioxidant, and most in particular from 0.5 mM to 25 mM antioxidant or even from 1 mM to 20 mM.

In particular, the Fc multimer composition may comprise from 0.01 mM to 100 mM methionine, in particular from 0.05 mM to 20 mM methionine, more in particular from 0.1 mM to 17 mM methionine, even more in particular from 0.5 mM to 15 mM methionine and most in particular from 1 mM to 12 mM methionine.

In a preferred embodiment, the Fc multimer composition according to the present invention further comprises an antioxidant, in particular methionine.

The composition according to the present invention may further comprise a surfactant. The term “surfactant” according to the present invention describes a compound that lowers the surface tension (or interfacial tension) between two liquids, between a gas and a liquid, or between a liquid and a solid. Thereby, a surfactant may when dissolved in water, lower the advancing contact angle, aid in displacing an air phase at the surface, and replaces it with a liquid phase.

Surfactants that may be used in an Fc multimer composition according to the present invention are well known to the person skilled in the art. In particular, any surfactant suitable for pharmaceutical applications, in particular suitable for being administered to a subject's body, such as a human patient, may be used.

For example, the surfactant may be one of the subclasses of anionic, nonionic, zwitterionic surfactants or may belong to the subclass of surfactants having cationic head groups.

In particular, a non-ionic surfactant may be used. Suitable nonionic surfactants are well known to the person skilled in the art, such as polyoxyethylene glycol octylphenol ethers (such as C8H17-(C6H4)-(O—C2H4)1-25-OH (e.g. Triton™ X-100)), sorbitan alkyl esters (such as SPAN® and/or polyoxyethylene glycol sorbitan alkyl esters, such as polysorbate (e.g. polysorbate 20 or polysorbate 80)) and/or poloxamers (such as Antarox®). In particular, a sorbitan alkyl ester, more in particular a polyoxyethylene glycol sorbitan alkyl ester, even more in particular a polysorbate and, most in particular polysorbate 80 is used.

Usually, also anionic surfactants may be used. Suitable anionic surfactants are well known to the person skilled in the art, such as dioctyl sodium sulfosuccinate (DOSS), Sodium lauryl ether sulfate (e.g. Texapon®), lignosulfonate and/or sodium stearate.

In general, the composition of the present invention may comprise one or more surfactants. If the composition comprises more than one surfactant, these may belong to the same or different subclasses of surfactants. Moreover, if the composition comprises more than one surfactant these may be any mixture of surfactants mentioned above.

Suitable amounts of surfactant that may be used in the present invention are well known to the person skilled in the art any may also depend on the type of the surfactant. In general, the composition may comprise from 0.0001% w/v to 0.5% w/v surfactant, in particular from 0.001% w/v to 0.1% w/v surfactant, more in particular from 0.005% w/v to 0.05% w/v surfactant and most in particular from 0.01% w/v to 0.03% w/v surfactant. For example, the composition may comprise from 0.0001% w/v to 0.5% w/v polysorbate 80, in particular from 0.001% w/v to 0.1% w/v polysorbate 80, more in particular from 0.005% w/v to 0.05% w/v polysorbate 80 and most in particular from 0.01% w/v to 0.03% w/v polysorbate 80.

In a preferred embodiment, the Fc multimer composition according to the present invention further comprises a surfactant, in particular polysorbate 80.

The Fc multimer composition according to the present invention may further comprise conventional pharmaceutical additives and adjuvants, excipients or diluents, including, but not limited to, water, salts, gelatin of any origin, vegetable gums, ligninsulfonate, talc, sugars, starch, gum arabic, vegetable oils, polyalkylene glycols, flavoring agents, preservatives, emulsifying agents, lubricants, colorants, wetting agents, fillers, and the like, such as other substances that do not react deleteriously with the active compounds. The Fc multimer composition may further comprise enzymes, such as protease inhibitors.

In particular, the Fc multimer composition according to the present invention may further comprise one or more salts. Suitable salts are well known to the person skilled in the art, in particular those suitable for pharmaceutical applications, such as sodium chloride. In particular, the Fc multimer composition according to the present invention may further comprise 1 mM to 250 mM salt, more in particular 10 mM to 200 mM salt, most in particular 30 mM to 170 mM salt.

Optionally, the suspension may also contain further suitable agents that increase the solubility of the compounds to allow for the preparation of highly concentrated solutions.

In one embodiment of the invention, the Fc multimer composition according to the present invention may comprise 70 mg/ml Fc multimer, in particular CSL730, L-histidine buffer, sodium chloride and L-proline, polysorbate 80, and water for injection.

The Fc multimer composition may be sterilized or treated in any way well known to the person skilled in the art to be suitable for preserving the composition for storage.

In a second aspect, the invention relates to an Fc multimer composition, comprising the Fc multimer composition of the invention in lyophilized form.

All embodiments described above with respect to Fc multimer composition according to the first aspect may also apply to the second aspect.

The term “lyophilized” according to the present invention means any low temperature dehydration process involving freezing the product, lowering pressure and removing the ice by sublimation.

The Fc multimer composition may be lyophilized by any lyophilization technique known to the person skilled in the art. Lyophilization may be performed by any means and at any temperature. Preferably, lyophilization is performed at room temperature (RT).

The Fc multimer composition may be pretreated before lyophilization, which may comprise e.g. concentrating of the Fc multimer composition.

The lyophilization may comprise a first phase of primary drying. During the primary drying phase, the pressure may be lowered (to the range of a few millibars), and enough heat may be supplied to the material for the ice to sublime. The amount of heat necessary may be calculated using the sublimating molecules' latent heat of sublimation. In this initial drying phase, about 95% of the water in the material may be sublimated. This phase may be slow (can be several days in the industry), because, if too much heat is added, the material's structure could be altered.

In the primary drying phase, pressure may be controlled through the application of partial vacuum. The vacuum usually speeds up the sublimation, making it useful as a deliberate drying process. Furthermore, a cold condenser chamber and/or condenser plates may provide a surface for the water vapour to reliquify and solidify on.

The lyophilization may comprise a second phase of secondary drying in which the temperature may be raised higher than in the primary drying phase, and can even be above 0° C. Usually the pressure may also be lowered in this stage to encourage desorption (typically in the range of microbars, or fractions of a pascal). However, there may be products that benefit from increased pressure as well. After lyophilization the buffer components may remain in form of dried salts.

Further, after the lyophilization process the material may be sealed, such as in a glass vial. In general, the resulting powder may be reconstituted by the addition of a suitable solvent prior use. The solution may be an aqueous solution, such as an aqueous buffered saline solution

Preferably, lyophilization is performed until (most of) the liquid is removed. At the end of the lyophilization process, the final residual water content in the product usually is extremely low. In particular, the final residual water content in the product is around 1% to 4%.

In particular, the lyophilizate described herein may be substantially pure and/or sterile.

In a preferred embodiment, in the Fc multimer composition in lyophilized form of the present invention, the Fc multimer is composed of 3 to 10 Fc portions.

In a more preferred embodiment, in the Fc multimer composition in lyophilized form of the present invention, the Fc multimer is composed of 3 Fc portions.

In a third aspect, the invention relates to the Fc multimer composition of the invention for use in the treatment of an autoimmune disease or an inflammatory disease.

The term “autoimmune disease” according to the present invention describes a condition arising from an abnormal immune response to a normal body part and is a subtype of inflammatory diseases.

In general, the Fc multimer composition of the invention may be used in the treatment of any autoimmune disease known to the person skilled in the art. In particular, the Fc multimer composition of the invention may be used in the treatment of any autoimmune disease known to the person skilled in the art to be curable by administration of Fc portions or Fc multimers. In general, the Fc multimer composition of the invention may be used in the treatment of an autoimmune disease in any body part.

Examples of autoimmune diseases that may be treated with the Fc multimer composition according to the present invention may include celiac disease, diabetes mellitus type 1, Graves' disease, inflammatory bowel disease, multiple sclerosis, psoriasis, rheumatoid arthritis, idiopathic thrombocytopenic purpura (ITP), Kawasaki's disease, Guillain-Barré syndrome (GBS), Chronic Inflammatory Demyelinating Polyneuropathy (CIDP), Multifocal Motor Neuropathy (MMN), myasthenia gravis, ulcerative colitis, immune-complex mediated kidney disorders, scleroderma, dermatomyositis, neuromyelitis optica, Sjogren's Syndrome, systemic vasculitis, or systemic lupus erythematosus.

The term “inflammatory disease” according to the present invention describes a disorder or condition characterized by inflammation, i.e. a complex biological and protective response of body cells and tissues to harmful stimuli (such as pathogens, damaged cells, or irritants). An inflammation usually involves immune cells, blood vessels, and molecular mediators.

Examples for an inflammatory disease are asthma, chronic peptic ulcer, tuberculosis, periodontitis, Crohn's disease, glomerulonephritis, transplant rejection, sinusitis or active hepatitis.

Further, all embodiments described above with respect to Fc multimer composition according to the first aspect also apply to the third aspect.

The use of the Fc multimer composition of the present invention in the treatment of an autoimmune disease or an inflammatory disease may comprise any form of administration known to the person skilled in the art, such as parenteral administration, e.g. subcutaneous, intravenous and/or transdermal administration. In particular, the use of the Fc multimer composition of the present invention in the treatment of an autoimmune disease or an inflammatory disease comprises its subcutaneous administration.

Further, the use of the Fc multimer composition of the present invention in the treatment of an autoimmune disease or an inflammatory disease may be accompanied by the parallel use of further immunoglobulins or parts thereof, nonsteroidal anti-inflammatory drugs (NSAIDs) and/or immunosuppressants. The Fc multimer composition and the further immunoglobulins or parts thereof, nonsteroidal anti-inflammatory drugs (NSAIDs) and/or immunosuppressants may be administered together in one administration or separately in two or more administrations.

The invention is not limited to the particular methodology, protocols and reagents described herein because they may vary. Furthermore, the terminology used herein is for the purpose of describing particular embodiments only and is not intended to limit the scope of the present invention. As used herein and in the appended claims, the singular forms “a”, “an”, and “the” include plural reference unless the context clearly dictates otherwise. Similarly, the words “comprise”, “contain” and “encompass” are to be interpreted inclusively rather than exclusively.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the preferred methods, and materials are described herein.

The present invention is further illustrated by the following Figures and Examples, which are intended to explain, but not to limit the invention, and from which further features, embodiments and advantages may be taken. As such, the specific modifications discussed are not to be construed as limitations on the scope of the invention. It will be apparent to the person skilled in the art that various equivalents, changes, and modifications may be made without departing from the scope of the invention, and it is thus to be understood that such equivalent embodiments are to be included herein.

### DESCRIPTION OF THE FIGURES

FIG. 1: Soluble (physical) aggregation (% HMW) over time at 35° C. for formulations 1-12

FIG. 2: Plot for pooled data for rates of aggregation at 25 and 35° C. as a function of measured pH

FIG. 3: Fragmentation (% LMW species) over time (NR-Caliper) at 35° C. for formulations 1-12

FIG. 4: Plot for pooled data for rates of fragmentation (±SE) at 35° C. as a function of measured pH

FIG. 5: Formation of acidic species over time (CEX) at 35° C. for all formulations

FIG. 6: Plot for pooled data for rates of formation of acidic species at 25 and 35° C. as a function of measured pH

FIG. 7: Subvisible particle analysis (a) Particle counts (particles/mL) for particles ≥10 microns (black bars) and for particles ≥25 microns (grey bars) after 3 months at 5° C. and 25° C. in F1, F2, F3, F4, F5, F9, F12, F17. (b) Overview of particle counts (particles/mL) for particles in all size ranges: 2-5 microns (black bars), 5-10 microns (grey bars), 10-25 microns (stripes across) and 10-25 microns (stripes diagonal) after 3 months at 5° C. and 25° C. in F1, F2, F3, F4, F5, F9, F12, F17.

FIG. 8: Initial (T0) results for % acidic and basic species as measured by cation exchange chromatography.

FIG. 9: Representative chromatogram from RP-HPLC to quantify oxidation of the Fc multimer molecule (CSL730) in a formulation

FIG. 10: Plot comparing initial (T0) % short chain oxidation species, long chain oxidation species and relative % total oxidation as measured by RP-HPLC for all formulations of the Fc multimer molecule (CSL730)

FIG. 11: Plot showing progress of % total oxidation, as measured by RP-HPLC, for all formulations of the Fc multimer molecule (CSL730) at 25° C. and 40° C. for up to 2 months of storage stability

FIG. 12: Illustration of an Fc construct containing three Fc domains formed from four polypeptides like CSL730. The first polypeptide (502) contains one Fc polypeptide containing different charged amino acids at the CH3-CH3 interface than the wt sequence (506) joined in a tandem series with a protuberance-containing Fc polypeptide (504). The second polypeptide (508) contains an Fc polypeptide containing different charged amino acids at the CH3-CH3 interface than the wt sequence (512) joined in a tandem series with another protuberance-containing Fc polypeptide (510). The third and fourth polypeptides (514 and 516, respectively) each contain a cavity-containing Fc polypeptide.

### EXAMPLES

Part 1—pH-Buffer-Salt Screening Study

To study the effect of pH, buffer type and ionic strength on formulations comprising an Fc multimer molecule (CSL730) at high concentrations, CSL730 formulations with a protein concentration of 100 mg/mL were prepared and investigated for protein stability.

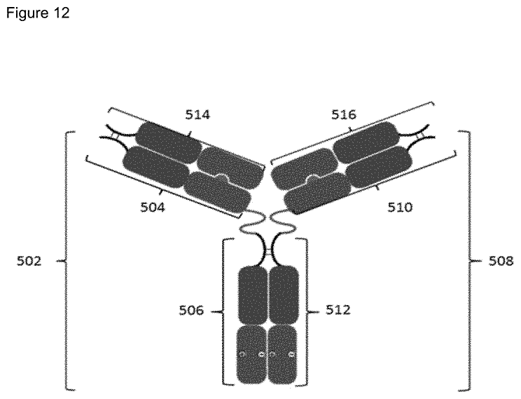

CSL730 is a trivalent Fc multimer, consisting of two long chains of SEQ ID NO: 24, and two short chains of SEQ ID NO: 23, produced essentially as described in WO 2015/168643 A2. This results in a trivalent Fc multimer as shown in FIG. 12.

### Materials and Methods

Materials: The materials used for the examples, their catalog numbers and the suppliers are listed in Table 1.

TABLE 1
Materials used for the examples

| | Catalog | |
|---|---|---|
| Material | Number | Supplier |
| 15 ml 10K molecular weight cut | 87731 | Life |
| off (MWCO) cassette | | Technologies |
| | | Australia |
| | | Pty Ltd |
| 2 ml liquid vials with 13 mm neck | BO2128 | Schott |
| L-Histidine | 1.043521.1000 | Merck |
| Sucrose | 1.00892.5000 | Merck |
| Sodium chloride | 1.06400.5000 | Merck |
| Citric acid monohydrate | 1.00244.500 | Merck |
| Acetic acid (glacial) | 1.00056.2500 | Merck |
| Amicon Ultra-15 Centrifugal Filter | UFC903024 | Merck |
| Devices (30 kDa) | | |
| Millex-GP Syringe Filter Unit, | SLGP033RS | Merck |
| 0.22 μm | | |
| Hydrochloric acid, 6M | 1.10164.1000 | Merck |
| Sodium hydroxide solution, 4N | 1048 | ACR Chemical |
| | | Reagents |

Preparation of formulations: The formulations were prepared with CSL730 (recombinant Fc multimer bulk purified protein (concentration: 146 mg/ml)) as starting material. The bulk purified protein was dialysed against each of the 12 buffers listed below. The pH of each buffer was measured after preparation. 30% overage (mg protein) was applied to account for losses. The formulations were dialysed against about 1 L of buffer for about 5 hours which was subsequently replaced with fresh buffer (about 1 L) and dialysed overnight. Dialysis occurred at 2-8° C. in 15 ml 10K MWCO cassettes. The following buffers were prepared for the dialysis:

1. 20 mM glacial acetic acid, 50 mM Sucrose, pH 4.5

2. 20 mM glacial acetic acid, 50 mM Sucrose, pH 5

3. 20 mM glacial acetic acid, 50 mM Sucrose, pH 5.5

4. 20 mM Histidine, 50 mM Sucrose at pH 5

5. 20 mM Histidine, 50 mM Sucrose at pH 5.5

6. 20 mM Histidine, 50 mM Sucrose at pH 6

7. 20 mM histidine, 50 mM Sucrose at pH 6.5

8. 20 mM Histidine, 50 mM Sucrose at pH 7

9. 20 mM Histidine, 50 mM Sucrose, 100 mM NaCl at pH 6

10. 20 mM citric acid monohydrate, 50 mM Sucrose, pH 5.5

11. 20 mM citric acid monohydrate, 50 mM Sucrose, pH 6

12. 50 mM Sucrose at pH 5.3 (buffer free)

After dialysis, the protein concentration of each sample was measured. When samples were found to be too dilute they were up-concentrated by centrifugation using Amicon tubes (30 kDa cut-off) with the aim of targeting 100±10 mg/mL. Protein concentration was confirmed afterwards.

Fill and finish was performed under a laminar flow hood. Each formulation was filtered through a 0.22 μm filter and filled into pre-labelled 2 ml glass vials with 1 ml of the formulation. Vials were then stoppered, and crimped.

A summary of the formulations prepared for this study are presented in Table 2. The target buffer concentration for all formulations was 20 mM. Acetate, histidine and citrate buffers were used at different respective pHs to achieve good buffer capacity within a pH range of 4.5-7. No surfactant was added to the formulations in this study. 50 mM Sucrose was added as a stabilizer to all formulations to provide a minimum level of stability. Note that sucrose does not impact ionic strength. One buffer-free formulation was prepared since proteins at high concentrations are known to be self-buffering. The pH of the buffer-free solution will depend on the protein itself.

TABLE 2
Summary of formulations prepared for the present study

| | | | Buffer | | | Sucrose |
|---|---|---|---|---|---|---|
| Formulation | Formulation | | Conc. | Target | NaCl | Conc. |
| No. | Buffer | pKa | (mM) | pH | (mM) | (mM) |
| 1 | acetate | 4.76 | 20 | 4.5 | — | 50 |
| 2 | | | 20 | 5.0 | — | 50 |
| 3 | | | 20 | 5.5 | — | 50 |
| 4 | histidine | 6.04 | 20 | 5.0 | — | 50 |
| 5 | | | 20 | 5.5 | — | 50 |
| 6 | | | 20 | 6.0 | — | 50 |
| 7 | | | 20 | 6.5 | — | 50 |
| 8 | | | 20 | 7.0 | — | 50 |
| 9 | | | 20 | 6.0 | 100 | 50 |
| 10 | citrate | 3.13 (1), | 20 | 5.5 | — | 50 |
| 11 | | 4.76 (2), | 20 | 6.0 | — | 50 |
| | | 6.40 (3) | | | | |
| 12 | buffer-free | N/A | — | molecule | — | 50 |
| | | | | dependent | | |

After filling, the filled formulations were placed in stability chambers at 2-8° C., 25° C. or 35° C. Samples were analyzed immediately after fill/finish (for “t0”/t=0 analysis) and at predetermined subsequent time points for up to 4 weeks. In order to compare kinetics of degradation between the different formulations, rate constants of degradation by different pathways (with standard error) were measured using multiple linear regression spanning a minimum of 3 time points (t=0, t=2 weeks and t=4 weeks). Multiple linear regression was done using a general linear model (GLM) to plot and fit the assay data to the model (equation 1).

% P=Pₒ+k·t [Equation 1]

### Methods Used for Analyzing the Stability of the Formulations:

Stability indicating methods were used to analyze the formulations at the different time points, namely: Size exclusion chromatography (SEC), Cation exchange chromatography (CEX) and Capillary Gel Electrophoresis (CGE) (Caliper). In addition, pH and visual appearance (for color, turbidity and visible particles) of the formulations were monitored at the different time points.

Methods used for the study, purpose of each method and analyses used at each analysis time point are summarized in Table 3.

TABLE 3
Analytical methods used for stability studies of formulations

| | | Time point (weeks) and storage | | |
|---|---|---|---|---|
| | | temperatures (° C.) | | |
| | | t0 (initial | 2 | 4 |
| Method | Purpose | time point) | weeks | weeks |
| Visual | Visible particles, | Initial | 25° C., | 2-8° C., |
| inspection | colour, opalescence, | | 35° C. | 25° C., |
| | turbidity | | | 35° C. |
| pH measurement | pH testing | Initial | 25° C., | 2-8° C., |
| | | | 35° C. | 25° C., |
| | | | | 35° C. |
| UV spectroscopy | Protein content | Initial | 25° C., | 2-8° C., |
| | (A280) | | 35° C. | 25° C., |
| | | | | 35° C. |
| Size exclusion | High molecular | Initial | 25° C., | 2-8° C., |
| chromatography | weight species | | 35° C. | 25° C., |
| (SEC) | (soluble aggregates) | | | 35° C. |
| Cation exchange | Acidic, basic and | Initial | 25° C., | 2-8° C., |
| chromatography | main species | | 35° C. | 25° C., |
| (CEX) | | | | 35° C. |
| Capillary Gel | Low molecular | Initial | 25° C., | 2-8° C., |
| Electrophoresis | weight species | | 35° C. | 25° C., |
| (CGE) (Caliper) | | | | 35° C. |

A description of the analytical methods is provided below:

Visual inspection: Visual inspection was conducted in an inspection station equipped with a white and black background and fluorescent light. Formulations in vials were gently swirled without producing bubbles then inspected for colour, clarity and the presence of visible particles. Inspections were conducted by two independent inspectors.

pH measurement: pH was measured using a Mettler Toledo SevenExcellence pH meter equipped with a InLab® Ultra Micro ISM electrode.

UV spectroscopy: Protein concentration was measured by using A280/UV determination on the formulations without dilution on an IMPLEN P360 Nanophotometer. Measurements were conducted 3 times per formulation and the mean values of the measurements calculated.

Size exclusion chromatography (SEC)-high performance liquid chromatography (HPLC): SEC-HPLC was used to determine the protein aggregation profile of the formulations. A Dionex system (Ultimate 3000) equipped with an Acquity BEH200 column (Waters, 4.6×150 mm) was used to analyse the samples. Samples were diluted to 10 g/L in appropriate buffer, 3.0 μL was injected and the separation was performed under isocratic conditions at a flow rate of 0.3 mL/min. Mobile phase consisted of BES buffer (pH 6.5) with a run time of 15 min. Intact protein was detected at 280 nm at a retention time of roughly 3.5 min. Monomer species, high molecular weight species (HMWS, aggregates) and low molecular weight species (LMWS, fragments) were reported as a relative area %. Internal and external references were used to validate the run.

Cation exchange chromatography (CEX): CEX-HPLC was used to determine the proportions of proteinaceous acidic, main and basics species. A Dionex system (Ultimate 3000) equipped with a Propack WCX-10 column (Thermo Fisher, 4×250 mm) was used to analyse the samples. Samples were diluted to 10 g/L in appropriate buffer, 3.0 μL injection volume was used and separation was conducted with a gradient method at 0.7 ml/min. Briefly, two aqueous buffers at pH 5 and pH 10 (Imidazole, Piperazine and TRIS based) were alternated over a period of 55 minutes. Species were detected at 280 nm, identified against a reference standard and reported as Relative Area percentage over the integrated area.

Capillary Gel Electrophoresis (CGE) “Caliper” method: The protein “banding pattern” was obtained by Capillary Gel Electrophoresis. Analysis was performed using a microfluidic LabChip GXII system (Perkin Elmer Australia Pty Ltd). The protein electrophoresis on the microfluidic chip was achieved by integration of the main features of one-dimensional SDS-PAGE: these include the separation, staining, de-staining, and detection. Denatured proteins were loaded onto the chip directly from a microtiter plate through a capillary sipper. The samples were then electrokinetically loaded and injected into the 14 mm long separation channel containing a low viscosity matrix of entangled polymer solution. The entire sample preparation procedures were done according to manufacturer protocol. For non-reducing samples, protein solution were diluted to 1 g/L with buffer and Milliq water. Reducing samples were diluted with 1M DTT. Denaturation occurred 40° C. for 20 min for non-reduced samples and at 80° C. for 15 min for reduced samples. Results were reported in relative area percentage for LMWS Intact and HMWS for non-reduced samples. For reduced samples, Long Chain and Short chain fraction were considered.

### Example 1—Visual Inspection at t=0

Visual appearance of all formulations directly before filling was evaluated. Additionally, all filled vials of all formulations were assessed by visual appearance after fill/finish.

Due to high protein concentration, all formulations possessed a brownish yellow colour with slight opalescence. Opalescence was observed to increase with pH. CSL730 in formulations at pH 6.5 and 7 precipitated with more intense precipitate seen at pH 7. It is well known that higher pH can be used to precipitate Fc fragments. The precipitated fragments can be recovered later on by solubilization in low pH buffers. This has been confirmed with the precipitated portion of the recombinant Fc multimer (data not shown). However, the impact of this high pH exposure on storage stability is not well understood.

All formulations were filtered before filling to vials (including the precipitated formulations) and reinspected after stoppering and crimping. All formulations had a brownish-yellow colour, were slightly opalescent and were free of visible particles (including pH 6.5 and pH 7.0 formulations). No gelling was observed in any of the formulations prepared.

Overall, results showed that formulations at pH 6.5 led to protein precipitation. Formulations with lower pH values down to pH 4.5 were more suitable since lower pHs did not lead to protein precipitation.

### Example 2—pH and Protein Concentration Measurements at t=0

pH and protein concentration measurements on all formulations 1-12 (see Table 2) were conducted at t=0. Differences from target values were also assessed. The results are summarized in Table 4

pH measurements showed that the measured pH values were within ±0.2 pH units for most formulations in comparison to the target pH-values, with the exception of the acetate formulation with the target pH 4.5 and the histidine formulation with the target pH 5.0, where the pH-values were within 0.4 and 0.3 units from the target pH, respectively. Overall, based on the measured pH values, the pH screening for this study covers a pH range of 4.88 to 6.92.

With regards to measured protein concentrations, most formulations were within the target concentration of 100+/−10 mg/mL. Exceptions were the acetate formulations with target pH 4.5 and 5.0. Those samples were over diluted by mistake, so this is considered a study deviation. On the other hand, histidine formulations at pH 6.5 and 7.0 had a lower concentration as a result of the precipitation that occurred.

The results show that the pH-value of the formulations was in most cases as expected. With the exception of the acetate formulations, a significant drop in protein concentration at pH 6.5 is associated with the significant precipitation of the protein under those conditions.

TABLE 4
Measured pH (and differences from target values) and
protein concentration values for all formulations.

| | | | | | Measured protein |
|---|---|---|---|---|---|
| Formulation | Formulation | Target | Measured | | concentration |
| No. | Buffer | pH | pH | ΔpH | (mg/mL)** |
| 1 | 20 mM Acetate | 4.5 | 4.9 | 0.4*** | 74 ± 1*** |
| 2 | | 5.0 | 5.2 | 0.2 | 72 ± 1*** |
| 3 | | 5.5 | 5.6 | 0.1 | 102 ± 1 |
| 4 | 20 mM Histidine | 5.0 | 5.3 | 0.3*** | 91 ± 1 |
| 5 | | 5.5 | 5.7 | 0.2 | 107 ± 3 |
| 6 | | 6.0 | 6.2 | 0.2 | 96 ± 5 |
| 7 | | 6.5 | 6.6 | 0.1 | 82 ± 6**** |
| 8 | | 7.0 | 6.9* | N/A | 19 ± 1**** |
| 9 | 20 mM Histidine/ | 6.0 | 6.0 | 0.1 | 98 ± 5 |
| | 150 mM NaCl | | | | |
| 10 | 20 mM Citrate | 5.5 | 5.5 | 0.1 | 106 ± 10 |
| 11 | | 6.0 | 5.9 | 0.1 | 91 ± 8 |
| 12 | Buffer free | TBD | 5.7 | N/A | 96 ± 4 |

*pH only measured after 4 weeks at cold storage,
**Target 100 ± 10 mg/mL,
***sample overdilution (study deviation),
****protein concentration decreased due to sample precipitation

### Example 3—High Molecular Weight (HMW) Profile at t=0 by Size Exclusion Chromatography

All formulations 1-12 (see Table 2) were analyzed by size exclusion chromatography for the quantitation of high molecular weight (soluble aggregates) species at t=0, as described above.

Soluble aggregates profile for all formulations showed relatively low % HMW in all formulations (overall <1.5%), with a rough trend showing that increasing pH is related to a higher % of HMW molecules (until the point of precipitation). Interestingly, the initial % HMW of the filtered solutions of the formulations after precipitation at pH 6.5 also fell below 1.5%. This indicates that while a pH 6.5 is not suitable for formulating the Fc multimer molecule at high protein concentrations, it could be suitable for formulating the recombinant Fc multimer molecule at lower protein concentrations.

### Example 4—Low Molecular Weight (LMW) Profile at t=0 by Capillary Gel Electrophoresis

All formulations 1-12 (see Table 2) were analyzed by non-reducing capillary gel electrophoresis using Caliper (NR Caliper) for the quantitation of low molecular weight species (fragmentation) at t=0, as described above.

All formulations, with the exception of formulation 8 (precipitated at pH ˜7), had a relatively low % LMW (1.5-2.0%). Overall, the data shows no proportional relation between the pH and % LMWs. However, the increase in % LMWs at pH 6.9 indicates that there may be a threshold pH above which more measurable increases in % LMWs occurs.

### Example 5—Acidic and Basic Species Profile at t=0 by Cation Exchange Chromatography (CEX)

All formulations were analyzed by cation exchange chromatography (CEX) for the quantitation of acidic and basic species at t=0, as described above.

With the exception of formulations 7 and 8, all formulations showed comparable levels of acidic species. The percentage of acidic species was lower in formulation 7 (52%) and lowest in formulation 8 (45%).

The data showed no relation between pH and initial % acidic species. Drop in initial % acidic species in histidine buffer at pH≥6.5 indicates there may be a threshold pH above which a significant decrease of acidic species is observed.

With the exception of formulations 7 and 8, all formulations showed comparable levels of basic species. In comparison with all formulations, the percentage of basic species was lower in formulations 7 and 8 (4%).

The data showed no relation between pH and initial % basic species. Drop in initial % basic species in histidine buffer at pH≥6.5 indicates there may be a threshold pH above which a decrease of basic species is observed.

However, the results of formulations 7 and 8 may be influenced by protein precipitation for both acidic and basic species, as described above.

### Summary of Examples 1 to 5

Based on analyses performed during processing and immediately after fill/finish (T=0), it can be concluded that formulations with pH≥6.5 may not be suitable for the formulation of Fc multimer molecules at high concentrations (≥100 mg/mL).

Nevertheless, all formulations were placed on stability to cover the kinetics of degradation over the range of pH values in all formulations.

### Example 6—Visual Inspection Over Time

Formulations 1 to 12 (see Table 2) were visually inspected after 2 and 4 weeks. The results are summarized in Table 5.

As described above, two inspectors (Insp 1 and Insp 2) examined visual appearance at each time point and temperature. There was a general alignment between examiners in most cases.

No changes were observed with regards to color and clarity for most formulations at all storage temperatures. Color of all formulations remained slight brown yellow while clarity remained slightly opalescent. Formulations at higher storage temperatures started showing visible particles, but the most significant formation of particles occurred at pH≥6.5. Interestingly, the formulation with salt showed no visible particles compared to the same formulation at pH 6.

The results indicate that particle formation at each time point of visual inspection was increased at a pH≥6.5 in comparison to samples having a lower pH.

TABLE 5
Results of visual appearance testing by the two inspectors in formulations stored
for 2 or 4 weeks as 2-8° C., 25° C. and 35° C.

| | | 2 weeks | | | | 4 weeks | | | | | |
|---|---|---|---|---|---|---|---|---|---|---|---|
| | Target | 25° C. | | 35° C. | | 2-8° C. | | 25° C. | | 35° C. | |
| Buffer | pH | Insp 1 | Insp 2 | Insp 1 | Insp 2 | Insp 1 | Insp 2 | Insp 1 | Insp 2 | Insp 1 | Insp 2 |
| 20 mM | 4.5 | 0 | 0 | <5 | <5 | <5 | <5 | 0 | 0 | 0 | 0 |
| Acetate | 5.0 | 0 | 0 | 0 | 0 | 0 | <5 | 0 | 0 | 0 | <5 |
| | 5.5 | 0 | <5 | 0 | 0 | 0 | 0 | <5 | <5 | 0 | <5 |
| 20 mM | 5.0 | 0 | 0 | 0 | <5 | 0 | <5 | <5 | <5 | <5 | 0 |
| Histidine | 5.5 | 0 | 0 | 0 | <5 | 0 | 0 | 0 | 0 | 0 | <5 |
| | 6.0 | 0 | 0 | <5 | <5 | 0 | <5 | 0 | 0 | 0 | 0 |
| | 6.5 | 0 | <5 | <5 | <5 | Countless | Countless | <5 | <5 | 0 | <5 |
| | 7.0 | ND | ND | 0 | <5 | <5 | Countless | <5 | <5 | 0 | <5 |
| His/NaCl | 6.0 | 0 | 0 | 0 | 0 | 0 | 0 | 0 | 0 | 0 | 0 |
| 20 mM | 5.5 | 0 | 5-10 | <5 | 0 | 0 | 0 | 0 | 0 | <5 | <5 |
| Citrate | 6.0 | 0 | 0 | <5 | <5 | 0 | 0 | <5 | 0 | <5 | <5 |
| Buffer free | TBD | 0 | <5 | 0 | 0 | 0 | <5 | 0 | <5 | 0 | <5 |

### Example 7—pH and Protein Concentrations Over Time

The pH and protein concentrations were monitored in formulations 1 to 12 (see Table 2) at 2-8, 25 and 35° C. for up to 4 weeks.

Overall, within experimental error, results showed minimum shifts in pH and protein conc. at 2-8, 25 and 35° C. for all formulations for up to 4 weeks of storage.

### Example 8—Relationship Between the Formation of High Molecular Weight (HMW) and pH/Buffer/Salt Over Time (Size Exclusion Chromatography)

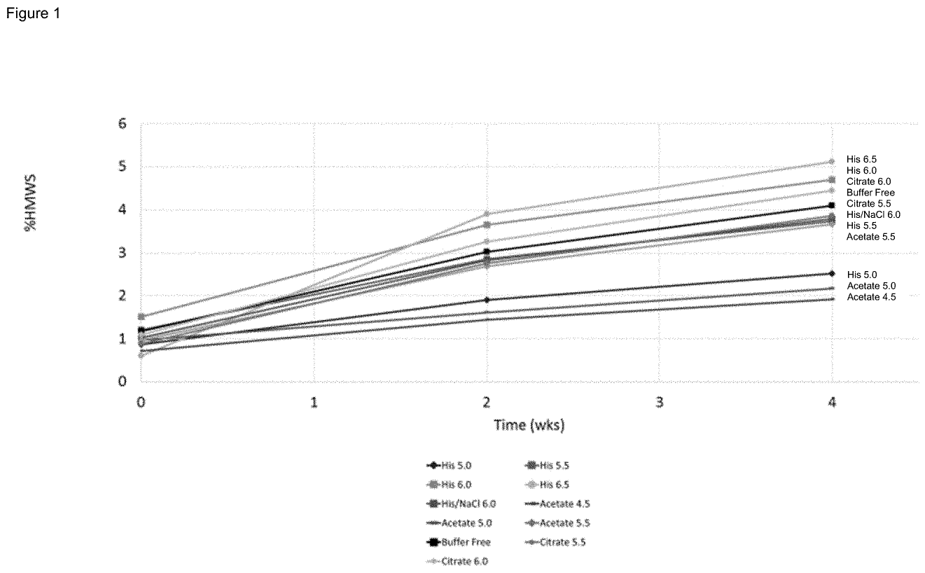

HMW species were monitored in formulations 1 to 12 (see Table 2) at 2-8, 25 and 35° C. for up to 4 weeks. Representative plots for changes in % HMW over time for formulations stored at 35° C. are shown in FIG. 1. FIG. 1 shows an initial high increase in % HMW species followed by a slower increase of species with time. The same trend was observed for the formulations stored at 25° C.

% changes in aggregate species (% HMW) after 4 weeks of storage at 2-8° C. are presented in Table 6. The aggregation rate constants, along with the corresponding standard error (SE), were determined for all formulations at 25 and 35° C. (from 3 time points) by multiple linear regression using Graphpad Prism software. The results are summarized in Table 6.

Results in Table 6 show that the rate of aggregation increased with temperature and with pH. Higher aggregation rates occurred at pH 6.0. The trend was more prominently observed at 35° C. Note that the formulation in histidine buffer with pH of 6.9 (formulation 8) appeared to be most stable. However, this is an artifact of the very low protein concentration in this formulation after protein precipitation (18 mg/mL), in comparison with much higher protein concentration in all other formulations (see Table 4).

The buffer type had a small impact on aggregation rate. At equivalent pH, different buffers performed similarly. The self-buffered formulation (formulation 12) was slightly less stable than the formulation with equivalent pH containing a buffer (formulation 5). The addition of salt (leading to a higher ionic strength) showed no significant impact on improvement in aggregation behaviour.

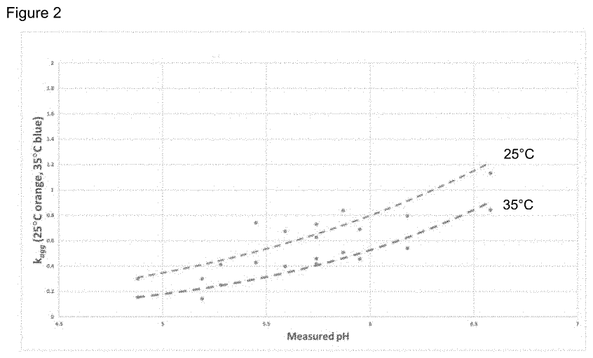

The trend for aggregation is summarized in FIG. 2 showing pooled data (aggregation rates at 25 and 35° C.) as a function of measured pH (without pH 7 formulation which is not representative of aggregation behavior). FIG. 2 shows overall a non-linear increase in the rate of aggregation as measured pH increased.

TABLE 6
Summary of SEC data for aggregation (% HMW) comparing changes in % HMW
after 4 weeks of storage at 2-8° C. (5^{th} column), aggregation rate constants
k (±SE) at 25° C. (6^{th} column) and at 35° C. (7^{th} column).

| | | | | Δ% HMW after | k at 25° C. | k at 35° C. |
|---|---|---|---|---|---|---|
| Formulation | | Target | Measured | T-4 weeks | (±SE) | (±SE) |
| No. | Buffer | pH | pH | (2-8° C.) | (%/week) | (%/week) |
| 1 | 20 mM | 4.5 | 4.9 | 0.1 | 0.2 ± 0.0 | 0.3 ± 0.0 |
| 2 | Acetate | 5.0 | 5.2 | 0.1 | 0.1 ± 0.0 | 0.3 ± 0.0 |
| 3 | | 5.5 | 5.6 | 0.7 | 0.4 ± 0.1 | 0.7 ± 0.1 |
| 4 | 20 mM | 5.0 | 5.3 | 0.4 | 0.3 ± 0.0 | 0.4 ± 0.1 |
| 5 | Histidine | 5.5 | 5.7 | 0.7 | 0.4 ± 0.1 | 0.6 ± 0.1 |
| 6 | | 6.0 | 6.2 | 1.0 | 0.5 ± 0.1 | 0.8 ± 0.2 |
| 7 | | 6.5 | 6.6 | 1.8 | 0.8 ± 0.3 | 1.1 ± 0.3 |
| 8 | | 7.0 | 6.9 | ~0 | 0.06* | 0.2 ± 0.0 |
| 9 | 20 mM | 6.0 | 6.0 | 0.8 | 0.5 ± 0.1 | 0.7 ± 0.1 |
| | Histidine/NaCl | | | | | |
| 10 | 20 mM | 5.5 | 5.5 | 0.5 | 0.4 ± 0.1 | 0.7 ± 0.1 |
| 11 | Citrate | 6.0 | 5.9 | 0.8 | 0.5 ± 0.1 | 0.8 ± 0.1 |
| 12 | Buffer free | TBD | 5.7 | 1.0 | 0.5 ± 0.1 | 0.7 ± 0.1 |

*Rate estimate from 2 points.

### Example 9—Relationship Between the Formation of Fragments (LMW) and pH/Buffer/Salt Over Time (Non Reducing Caliper)

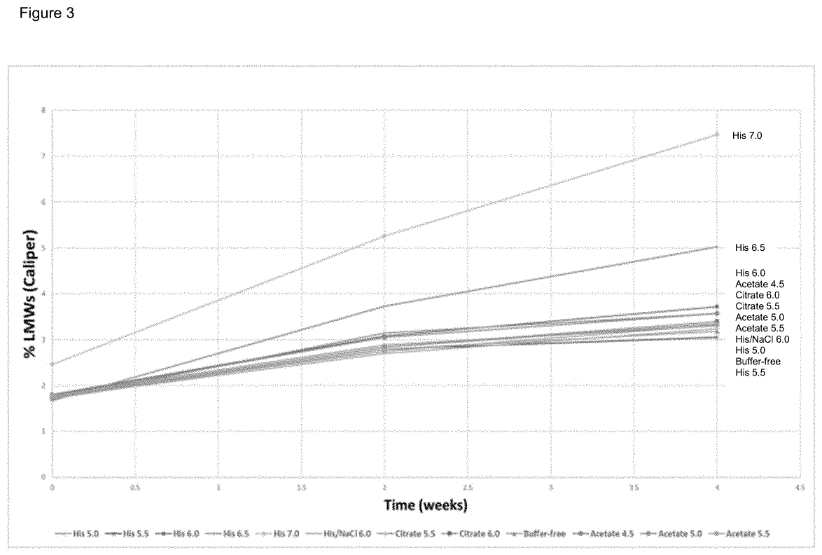

Fragmentation of formulations 1 to 12 (see Table 2) was monitored at 2-8, 25 and 35° C. for up to 4 weeks. Representative plots for changes in % fragments over time for formulations stored at 35° C. are shown in FIG. 3. FIG. 3 shows an initial high increase in % LMW species (fragments) followed by a slower increase of species with time. The same trend was observed for the formulations stored at 25° C.

% changes in fragments after 4 weeks of storage at 2-8° C. are presented in Table 7. Rates constants of fragmentation, along with the corresponding standard error (SE), were determined for the formulations at 25 and 35° C. (from 3 time points) by multiple linear regression using Graphpad Prism software. Multiple linear regression was done using a general linear model (GLM) to plot and fit the assay data to the model (equation 1). The results are summarized in Table 7.

The data revealed that generally low fragmentation occurs at 2-8° C. However, the rate of fragmentation increased with storage temperature and with pH (pH≥6.0). More significant changes in % LMW were observed at 2-8° C. with the formulation at ˜pH 7. At 25° C. and 35° C. the rate of fragmentation was also highest in formulation 8 at ˜pH 7 (despite lowest protein concentration at 18 mg/mL) followed by pH 6.5. At the same pH, the buffer type only had a small impact on aggregation rate. No clear benefits were found for the addition of NaCl (higher ionic strength).

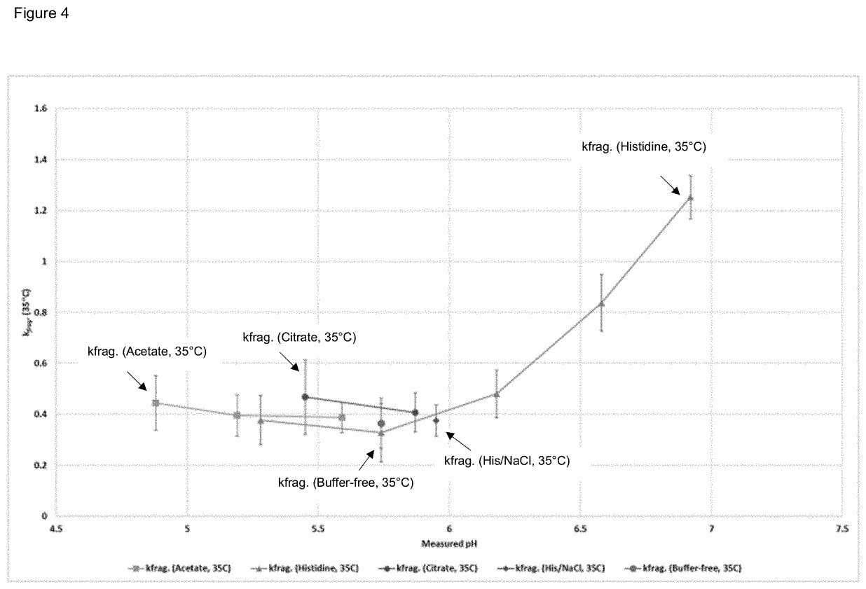

The trend for fragmentation is summarized in FIG. 4 showing pooled data (fragmentation rates at 35° C. with standard errors) as a function of measured pH. As was observed with aggregation, FIG. 4 shows overall that the rate of fragmentation increased as measured pH increased pH (≥6.0).

TABLE 7
Summary of NR-Caliper data for fragmentation (% LMW) comparing changes in
fragments after 4 weeks of storage at 2-8° C. (5^{th} column), fragmentation
rate constants k (±SE) at 25° C. (6^{th} column) and at 35° C. (7^{th} column).

| | | | | Δ% fragments | k at 25° C. | k at 35° C. |
|---|---|---|---|---|---|---|
| Sample | | Target | Measured | after T-4 | (±SE)* | (±SE)* |
| No. | Buffer | pH | pH | weeks (2-8° C.) | (%/wk) | (%/wk) |
| 1 | 20 mM | 4.5 | 4.9 | 0.1 | 0.2 ± 0.1 | 0.5 ± 0.1 |
| 2 | Acetate | 5.0 | 5.2 | 0.1 | 0.1 ± 0.1 | 0.4 ± 0.1 |
| 3 | | 5.5 | 5.6 | 0.1 | 0.1 ± 0.1 | 0.4 ± 0.1 |
| 4 | 20 mM | 5.0 | 5.3 | 0.1 | 0.1 ± 0.1 | 0.4 ± 0.1 |
| 5 | Histidine | 5.5 | 5.7 | 0.1 | 0.1 ± 0.1 | 0.3 ± 0.1 |
| 6 | | 6.0 | 6.2 | ~0 | 0.2 ± 0.1 | 0.5 ± 0.1 |
| 7 | | 6.5 | 6.6 | 0.1 | 0.3 ± 0.1 | 0.8 ± 0.1 |
| 8 | | 7.0 | 6.9 | 0.3 | 0.5* | 1.3 ± 0.1 |
| 9 | 20 mM | 6.0 | 6.0 | ~0 | 0.1 ± 0.1 | 0.4 ± 0.1 |
| | Histidine/NaCl | | | | | |
| 10 | 20 mM | 5.5 | 5.5 | 0.1 | 0.2 ± 0.1 | 0.8 ± 0.2 |
| 11 | Citrate | 6.0 | 5.9 | 0.1 | 0.1 ± 0.1 | 0.4 ± 0.1 |
| 12 | Buffer free | TBD | 5.7 | 0.1 | 0.1 ± 0.1 | 0.4 ± 0.1 |

*Rate estimate from 2 points.

### Example 10—Relationship Between the Formation of Acidic Species and pH/Buffer/Salt Over Time (Cation Exchange Chromatography)

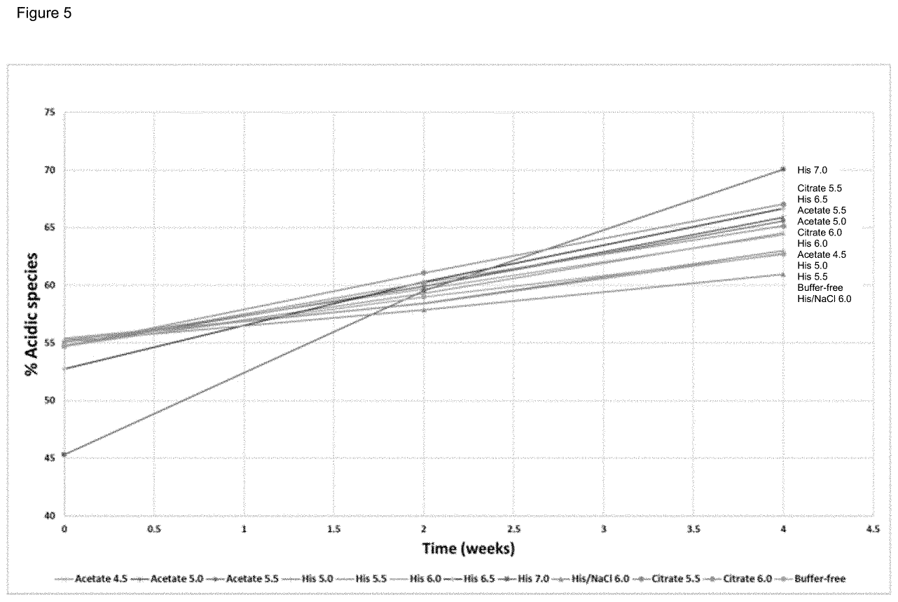

The formation of acidic species was monitored in formulations 1 to 12 (see Table 2) at 2-8, 25 and 35° C. for up to 4 weeks. Representative plots for changes in % acidic species over time for formulations stored at 35° C. are shown in FIG. 5. FIG. 5 shows (except for the histidine formulation at ˜pH 7) a more linear increase in % acidic species over time, as compared to plots for % HMW (FIG. 1) and % LMW (FIG. 3). The same trend was also observed for the formulations stored at 25° C.

% changes in acidic species after 4 weeks of storage at 2-8° C. are presented in Table 8. Rate constants of acidic species formation, along with the corresponding standard error (SE), were determined for the formulations at 25 and 35° C. (from 3 time points) by multiple linear regression using Graphpad Prism software. Multiple linear regression was done using a general linear model (GLM) to plot and fit the assay data to the model (equation 1). The results are also summarized in Table 8.

The data revealed that generally low fragmentation occurred at 2-8° C. However, the rate of formation of acidic species increased with storage temperature and with pH (pH≥6.0). The highest levels of acidic species formed after 4 weeks at 2-8° C. in the formulation at ˜pH 7. At 25° C. and 35° C. the rate of formation of acidic species was also highest in formulation 8 at ˜pH 7 (despite lowest protein concentration at 18 mg/mL) followed by pH 6.5.

At the same pH, the buffer type was also found to play a role in the rate of formation of acidic species, with formulations in histidine buffer showing more storage stability, as compared to the formulations in the same pH in acetate or citrate buffers.

The addition of NaCl (higher ionic strength) showed better storage stability, with regards to rate of formation of acidic species, than the formulation at the same pH without salt.

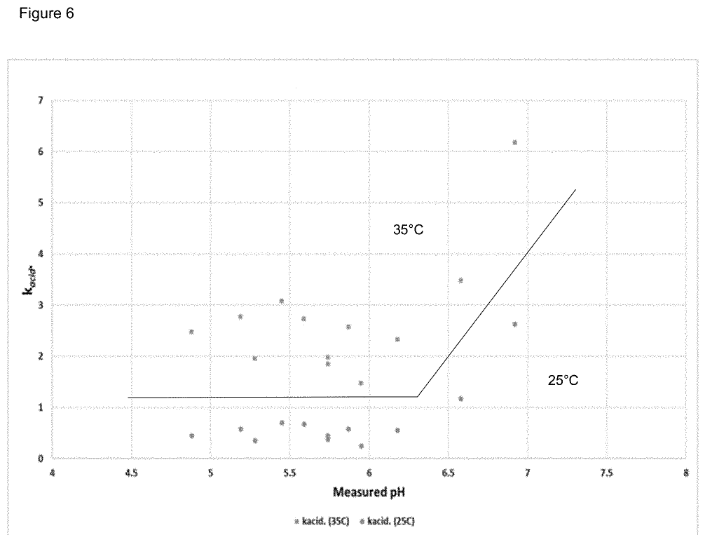

The trend for formation of acidic species with pH is summarized in FIG. 6 showing pooled data (rates of formation of acidic species at 25 and 35° C.) as a function of measured pH. As was observed with aggregation and fragmentation, FIG. 6 shows overall that the rate of formation of acidic species increased as measured pH increased (>pH 6.0).

TABLE 8
Summary of CEX data (% acidic species) comparing changes in % acidic species
after 4 weeks of storage at 2-8° C. (5^{th} column) and rate constants of acidic
species formation k (±SE) at 25° C. (6^{th} column) and at 35° C. (7^{th} column).

| | | | | Δ% acidic | | |
|---|---|---|---|---|---|---|
| | | | | species | k at 25° C. | k at 35° C. |
| Formulation | | Target | Measured | after T-4 wks | (±SE)* | (±SE)* |
| No. | Buffer | pH | pH | (2-8° C.) | (%/wk) | (%/wk) |
| 1 | 20 mM | 4.5 | 4.9 | 0.2 | 0.5 ± 0.1 | 2.5 ± 0.1 |
| 2 | Acetate | 5.0 | 5.2 | 0.2 | 0.6 ± 0.1 | 2.8 ± 0.1 |
| 3 | | 5.5 | 5.6 | 0.3 | 0.7 ± 0.0 | 2.7 ± 0.1 |
| 4 | 20 mM | 5.0 | 5.3 | 0.1 | 0.4 ± 0.1 | 2.0 ± 0.2 |
| 5 | Histidine | 5.5 | 5.7 | ~0 | 0.4 ± 0.1 | 1.9 ± 0.2 |
| 6 | | 6.0 | 6.2 | 0.3 | 0.6 ± 0.1 | 2.3 ± 0.0 |
| 7 | | 6.5 | 6.6 | ~0 | 1.2 ± 0.2 | 3.5 ± 0.2 |
| 8 | | 7.0 | 6.9 | 1.7 | 2.6* | 6.2 ± 0.5 |
| 9 | 20 mM | 6.0 | 6.0 | 0.4 | 0.3 ± 0.1 | 1.5 ± 0.0 |
| | Histidine/NaCl | | | | | |
| 10 | 20 mM | 5.5 | 5.5 | 0.1 | 0.7 ± 0.1 | 3.1 ± 0.1 |
| 11 | Citrate | 6.0 | 5.9 | 0.2 | 0.6 ± 0.1 | 2.6 ± 0.1 |
| 12 | Buffer free | TBD | 5.7 | 0.5 | 0.5 ± 0.1 | 2.0 ± 0.1 |

*Rate estimate from 2 points

### Conclusion of Examples 1 to 10

The results indicate that formulations of Fc multimer molecules (CSL730) at high concentrations (100 mg/mL) require specific conditions to prevent protein precipitation. It was shown that a pH≥6.5 may not be suitable for formulations of Fc multimer molecules (CSL730) at high concentrations (100 mg/mL) due to precipitation. Formulations of Fc multimer molecules (CSL730) at high protein concentrations (100 mg/mL) were more stable to aggregation, fragmentation and acidic species formation at pH lower than 6. Furthermore formulations of Fc multimer molecules (CSL730) at high protein concentrations (100 mg/mL) were more stable to acidic species formation with high salt concentration, as well as with histidine buffer.

### Part 2—Stabilizer Screening Study

Excipient screening studies were performed to investigate the effect of stabilizer type (sugars/polyols and amino acids), stabilizer levels, surfactant levels, antioxidant type and antioxidant levels on liquid formulations comprising an Fc multimer molecule (CSL730) at protein concentrations of 10-100 mg/ml. A parallel short term (3 month) study was conducted to investigate storage stability as a function of protein concentration. Overall, CSL730 formulations with protein concentrations ranging from 10-100 mg/mL were prepared and investigated for protein stability.

### Materials and Methods

Materials: The materials used for the examples were the same as in Part 1; additional materials used in this part, their catalogue numbers and the suppliers are listed in Table 9.

TABLE 9
Materials used for the examples, the corresponding
catalog number from the supplier and the supplier.

| | Catalog | |
|---|---|---|
| Material | Number | Supplier |
| Slide-A-Lyzer ® G2 Dialysis | 87732 | Thermofisher |
| Cassettes 10K MWCO, 30 ml | | Scientific |
| Slide-A-Lyzer ® G2 Dialysis | 87733 | Thermofisher |
| Cassettes 10K MWCO, 70 ml | | Scientific |
| 2 ml Fiolax clear vials with 13 | BO2128 | Schott |
| mm neck | | |
| 13 mm Injection Stopper | 19700004 | West |
| FluroTec | | Pharmaceutical |
| | | Services |
| 13 mm Flip Off Vial Seals | F013DBL-1K | West |
| | | Pharmaceutical |
| | | Services |
| L-Proline | 1.07430.1000 | Merck |
| L-arginine monohydrochloride | 1.01544.1000 | Merck |
| D (+) Trehalose dihydrate | T9531-100G | Sigma |
| D-Sorbitol | S1876 | Sigma |
| Polysorbate 80 | 7815 | NOF |
| L-glutathione reduced | G4251 | Sigma |
| L-Methionine | M8439 | Sigma |

Preparation of formulations: The formulations were prepared from CSL730 recombinant Fc multimer bulk purified protein at 120 mg/mL formulated in 20 mM Histidine, 40 mM NaCl and 200 mM Proline at pH 6.0.

Histidine was the buffer of choice in all formulations at a target buffer concentration of 20 mM. pH was fixed in all but one formulation to 5.25±0.1. Polysorbate 80 (PS80) was the surfactant of choice for all formulations. PS80 concentration was fixed in all but one formulation to 0.02 w/v. Different stabilizers were used (proline, arginine, sucrose, trehalose and sorbitol) spanning a target concentration range of 200-300 mM. Four formulations contained varying levels (1-20 mM) of antioxidants (either methionine or reduced glutathione GSH). Study (and hence formulation differences) was designed to primarily change one factor at a time (OFAT). A summary of the formulations prepared for this study is provided in Table 10.

The following buffers were prepared for the dialysis:
1. 20 mM histidine, 250 mM sucrose, pH 5.25
2. 20 mM histidine, 250 mM trehalose, pH 5.25
3. 20 mM histidine, 250 mM sorbitol, pH 5.25
4. 20 mM histidine, 250 mM arginine, pH 5.25
5. 20 mM histidine, 167 mM arginine, 83 mM sucrose, pH 5.25
6. 20 mM histidine, 225 mM sucrose, pH 5.25

To prepare the formulations (2-6, 8-16, 18), approximately 30-50 mL portions of the bulk purified protein were dialysed against each of the 6 buffers listed above. The pH of each buffer was measured after preparation. 30% overage (mg protein) was applied to account for losses. The formulations were dialysed against about 1 L of buffer for about 5 hours which was subsequently replaced with fresh buffer (about 1 L) and dialysed overnight. Dialysis occurred at 2-8° C. in 30 or 70 ml 10K MWCO cassettes.

After dialysis, the protein concentration of each dialysate was measured. When samples were found to be too dilute, they were concentrated up by centrifugation using Amicon tubes with the aim of targeting 120±5 mg/mL. Protein concentration was confirmed afterwards.

The following stock solutions were prepared and used as spiking solutions to aid in the preparation of the final formulations:
1. 20 mM histidine, 5.75% w/v (500 mM) proline, 0.02% w/v PS80, pH 5.25
2. 20 mM histidine, 17.04% w/v sucrose, 0.02% w/v PS80, pH 5.25
3. 10% w/v PS80
4. 20 mM histidine, 200 mM proline, 40 mM sodium chloride, 0.02% w/v PS80, pH 5.8 (labelled “CSL730 Diluent”)

Drug substance (DS) was composed of 118 mg/mL recombinant Fc multimer CSL730, 20 mM Histidine, 40 mM NaCl, 200 mM Proline and 0.02% w/v PS80 at pH 6.0. The DS was diluted by the addition of appropriate volumes of stock solution 1 or of stock solution 2 to achieve protein concentrations of 100±5 mg/mL followed by adjusting pH to 5.25±0.1 using 0.1M HCl to prepare formulation 1 and formulation 7, respectively. Formulation 17 was prepared by direct dilution of the DS to 100±5 mg/mL using stock solution 4 while maintaining the pH at 5.8±0.1.

After an up-concentration step and confirmation of protein concentration (A280 measurements), formulations 2-6, 8-16 and 18 were appropriately diluted with buffers 1-6, as needed, and final pH was adjusted to 5.25±0.1 using 0.1 M HCl. Protein concentration was confirmed after pH adjustment step.

Fill and finish was performed under a laminar flow hood. Each formulation and its corresponding placebo was filtered through a 0.22 μm PES sterile filter and filled into pre-labelled 2 ml glass vials with 0.8 ml of the formulation. Vials were then stoppered and crimped. In total, 17 liquid formulations and 13 placebos were prepared.

After fill/finish, the vials of the filled liquid formulations were placed in stability chambers at 2-8° C., 25° C. or 40° C. For “T0”/T=0 analysis samples were analyzed immediately after fill/finish followed by analysis at predetermined subsequent time points for up to 6 months. In order to compare kinetics of degradation between the different formulations, rate constants of degradation by different pathways (with standard error) were measured using multiple linear regression using JMP and Excel software. Multiple linear regression was calculated using a general linear model (GLM) to plot and fit the assay data to the model (equation 1).

% P=Po+k·t [Equation 1]

TABLE 10
Summary of formulations prepared for the excipient screening study

| | Target | Target | | | Stabilizer | Antioxidant | |
|---|---|---|---|---|---|---|---|
| Formulation | CSL730 conc. | PS80 conc. | Target | Stabilizer | Conc. | type and conc. | Molar ratio |
| No. | (mg/mL) | (% w/v) | pH | type | (mM) | (mM) | (protein:stabilizer) |
| F1* | 100 ± 5 | 0.02 | 5.25 ± 0.1 | Proline | 250 | | 1:417 |
| F2 | 100 ± 5 | 0.02 | 5.25 ± 0.1 | Sucrose | 250 | | 1:417 |
| F3 | 100 ± 5 | 0.02 | 5.25 ± 0.1 | Trehalose | 250 | | 1:417 |
| F4 | 100 ± 5 | 0.02 | 5.25 ± 0.1 | Sorbitol | 250 | | 1:417 |
| F5 | 100 ± 5 | 0.02 | 5.25 ± 0.1 | Arginine | 250 | | 1:417 |
| F6 | 100 ± 5 | 0.02 | 5.25 ± 0.1 | Sucrose/Arginine | 83/167 | | 1:417 |
| F7* | 100 ± 5 | 0.02 | 5.25 ± 0.1 | Sucrose/Proline | 83/167 | | 1:417 |
| F8 | 100 ± 5 | 0.02 | 5.25 ± 0.1 | Sucrose | 225 | | 1:375 |
| F9 | 100 ± 5 | 0.02 | 5.25 ± 0.1 | Sucrose | 300 | | 1:500 |
| F10 | 70 | 0.02 | 5.25 ± 0.1 | Sucrose | 250 | | 1:625 |
| F11 | 10 | 0.02 | 5.25 ± 0.1 | Sucrose | 250 | | 1:4167 |
| F12 | 100 ± 5 | 0.02 | 5.25 ± 0.1 | Sucrose | 250 | 10 mM | 1:417 |
| | | | | | | Methionine | |
| F13 | 100 ± 5 | 0.02 | 5.25 ± 0.1 | Sucrose | 250 | 20 mM | 1:417 |
| | | | | | | Methionine | |
| F14 | 100 ± 5 | 0.02 | 5.25 ± 0.1 | Sucrose | 250 | 1 mM | 1:417 |
| | | | | | | reduced GSH | |
| F15 | 100 ± 5 | 0.02 | 5.25 ± 0.1 | Sucrose | 250 | 10 mM | 1:417 |
| | | | | | | reduced GSH | |
| F17* | 100 ± 5 | 0.02 | 525 ± 0.1 | Proline | 200 | | 1:333 |
| F18 | 100 ± 5 | 0.04 | 5.25 ± 0.1 | Sucrose | 250 | | 1:417 |

*F1, F7 and F17 were the only formulation not dialysed. Thus, the formulations had low levels of NaCl (~30-40 mM).

### Methods Used for Analyzing the Stability of the Formulations:

Stability indicating methods were used to analyze the formulations at the different time points namely: Size exclusion chromatography (SEC), Cation exchange chromatography (CEX), Capillary Gel Electrophoresis (CGE) (Caliper) and reverse phase HPLC (RP-HPLC) for oxidation. In addition, pH, visual appearance (for color, turbidity and visible particles) and subvisible particles (SVP) of the formulations were monitored at the different time points.

Methods used for the study and purpose of each method are summarized in Table 11. Storage conditions and analysis time points are summarized in Table 12.

TABLE 11
Analytical methods used for stability studies
of formulations and their purpose

| Method | Purpose |
|---|---|
| Visual inspection | Visible particles, colour, |
| | opalescence, turbidity |
| Dynamic Imaging Particle Analysis | Sub-visible particle count |
| (DIPA) | |
| pH measurement | pH testing |
| Osmolality | Osmolality measurement |
| Polysorbate 80 (PS80) testing | Quantitation of PS80 |
| UV spectroscopy | Protein content (A280) |
| Size exclusion chromatography (SEC) | High molecular weight species |
| | (soluble aggregates) |
| Cation exchange chromatography | Acidic, basic and main species |
| (CEX) | |
| Capillary Gel Electrophoresis | Low molecular weight species |
| (CGE) (Caliper) | |
| Reverse phase high performance | Quantitation of Oxidation |
| liquid chromatography (RP-HPLC) | |

TABLE 12
A summary of storage conditions and analysis time points

| Storage condition | Time points | |
|---|---|---|
| 2-8° C. | 0, 3, 6 | months |
| 25° C. | 0, 1, 2, 3, 6 | months |
| 40° C. | 0, 1, 2, 3 | months |

A description of the analytical methods is provided below:

Visual inspection, pH measurement, UV spectroscopy, size exclusion chromatography, cation exchange chromatography, and capillary gel electrophoresis were carried out as described in Part 1 above.

Osmolality: Osmolality of the formulations was measured at the initial time point (T0) by freezing point depression using a Gonotec Osmomat 3000 Osmometer. Sample volumes were 300 μL. Measurements were conducted 3 times per formulation and the mean values of the measurements calculated.

Subvisible particle count testing: Limited analysis of subvisible particles using a FlowCam Biologics instrument (a Dynamic/flow Imaging Particle Analysis—DIPA—technique) was performed on formulations of interest at 2-8° C. and 25° C. only after 3 months storage time. A minimum sample volume of 0.5 mL was used. Measurements were conducted 3 times per formulation and the mean values of the measurements calculated.

Analysis of oxidation by reverse phase high performance liquid chromatography (RP-HPLC): A RP-HPLC method was used to determine the total amount of oxidized species as a percentage of the total area, and the relative amount of oxidation of both short and long chain. Sample preparation involved sample dilution to 10 g/L in appropriate buffer followed by the use of guanidine as denaturing reagent and of DL-Dithiothreitol (DTT) for the reduction of disulphide bonds. This step generates two identical long chains and two identical short chains. To prevent oxidation of the fragments, a subsequent dilution step with a solution containing high concentration of L-methionine was performed followed by sample analysis at a sample concentration of 2 μg/μl. A Thermo Ultimate 3000 (or equivalent) equipped with an AdvanceBio RP-mAb Diphenyl 3.5 μm, 2.1×50 mm column was used to analyse the samples. 3 μL injection volume was used and separation was conducted with a gradient method at 0.35 ml/min. Column temperature was set to 65° C. Briefly, two buffers (0.1% trifluoroacetic acid in water and 0.08% TFA in acetonitrile) were alternated over a period of 20 minutes. Species were detected at 280 nm, identified against a reference standard and reported as Relative Area percentage over the integrated area.

Analysis of polysorbate 80 (PS80): RP-HPLC was used to quantify the amount of PS80 at the initial time point (T0) in the different formulations. In short, PS80 standard and the samples were treated with ethanol followed by 0.1M KOH at 40° C. followed by sample analysis of oleic acid resulting from hydrolysis by a reverse phase HPLC method. A Dionex (Ultimate 3000) System (or equivalent) equipped with a Nova-Pak® C18 3.9×150 mm, 4 μm reverse phase column (Waters) was used to analyze the samples. Injection volume was 15 μL and separation was conducted using an isocratic method at 2.0 ml/min. Mobile phase was 80% acetonitrile with 20% potassium dihydrogen phosphate buffer at pH 2.8. Column temperature was set to 40° C. Species were detected at 250 nm, and quantified using a standard calibration curve generated by the PS80 standard solutions. Data is reported as (w/v) of PS80.

### Example 1—Visual Inspection at T0 and Over Time

All filled vials were examined by visual appearance after fill/finish (100% visual inspection) by two inspectors (Insp 1 and Insp 2, as described above) and at each time point and temperature. No significant discrepancies were found in the visual description between examiners.

Except for the 10 mg/ml formulation, all other formulations possessed a slight brownish yellow (BY) (70 mg/mL formulation) to BY (100 mg/mL formulations) colour with slight opalescence due to high protein concentration. 0-2 visible particles were observed randomly in some formulations and placebos. A close examination, with knowledge about the protein, revealed that the visible particles were exogenous in nature (probably introduced during the fill/finish process). Formulations at high protein concentrations did not undergo gelling.

Formulations 1 to 18 (see Table 10) were visually inspected for up to 6 months at different temperatures. The color of most formulations remained unchanged at all storage temperatures from T0 (colorless for 10 mg/ml formulation, slight BY for 70 mg/ml formulation and BY for 100 mg/ml formulations). Formulations with reduced GSH had a slightly more BY color, as compared to formulations at the same protein concentration. Clarity remained unchanged (clear in 10 mg/ml formulation and slightly opalescent in all others). Particle counts remained unchanged from T0 (0-2), regardless of temperature and time. None of the formulations gelled during the storage time period at any temperature.

With time, GSH-containing placebos exhibited discoloration to a slight BY or BY colour. The colour intensified with storage time, storage temperature and with increasing GSH concentration. Placebos for other formulations remained clear and colourless regardless of temperature and time. Overall, particle counts remained unchanged from T0.

### Example 2—Osmolality, pH, Protein Concentration and Polysorbate 80 Measurements at T0 and Over Time

Osmolality, pH, protein concentration and polysorbate 80 (PS80) concentration measurements on all formulations 1-18 (see Table 10) were conducted at the initial time point (T0). The results are summarized in Table 13.

The results in Table 13 show that protein concentration was within 100±5 mg/mL for the high concentration formulations. pH measurements showed that the measured pH values were within ±0.1 pH units for all formulations in comparison to the target pH-values. Osmolality measurements were generally within 300-390 mOsm/kg, with the exception of arginine-containing formulations (formulations 5, 6) where osmolality values were above 400 mOsm/kg. PS80 concentrations in all formulations were within a maximum of ±0.003 w/v of the PS80 target concentrations.

The pH and protein concentrations were monitored in formulations 1 to 18 (see Table 10) at 2-8, 25 and 40° C. for up to 6 months.

Table 14 summarizes results of pH measurements at different storage temperatures and times. Within experimental error, results showed minimum shifts (within a maximum of ±0.1 units) in pH at all temperatures for all formulations within the time frame measured.

Table 15 summarizes results of protein concentration measurements at different storage temperatures and times. Within experimental error, results showed small shifts in protein concentration with time. Three formulations showed relatively larger variations in protein concentration with time (F3 at 40° C. and F15 at 25° C. and 40° C.), but no protein precipitation appeared in any of these formulations. Average variation in protein concentration was 1.0 mg/mL at 2-8° C., 1.7 mg/mL at 25° C. and 1.6 mg/mL at 40° C. within the period measured. This is presumed to be a result of relatively assay variability of the high throughput technique used.

Table 13: Measured protein concentration (±standard deviation),
pH, osmolality (±standard deviation) and polysorbate 80 (PS80)
concentration for all formulations at initial time point (T0)

| | Target | Measured | | | | |
|---|---|---|---|---|---|---|
| | protein | protein | | | Measured | PS80 |
| Form. | conc. | conc. | Target | Measured | Osmo. | conc. |
| Code | (mg/mL) | (mg/mL) | pH | pH | (mOsm/kg) | (% w/v) |
| F1 | 100 | 102.8 ± 2.2 | 5.25 | 5.25 | 343 ± 2 | 0.019 |
| F2 | 100 | 95.9 ± 1.1 | 5.25 | 5.20 | 298 ± 2 | 0.020 |
| F3 | 100 | 100.7 ± 0.6 | 5.25 | 5.13 | 308 ± 3 | 0.019 |
| F4 | 100 | 97.5 ± 0.0 | 5.25 | 5.18 | 297 ± 4 | 0.020 |
| F5 | 100 | 99.2 ± 2.8 | 5.25 | 5.18 | 473 ± 2 | 0.021 |
| F6 | 100 | 103.8 ± 1.3 | 5.25 | 5.12 | 424 ± 5 | 0.020 |
| F7 | 100 | 100.5 ± 1.9 | 5.25 | 5.20 | 389 ± 4 | 0.017 |
| F8 | 100 | 102.3 ± 0.5 | 5.25 | 5.18 | 285 ± 1 | 0.019 |
| F9 | 100 | 95.2 ± 0.6 | 5.25 | 5.20 | 364 ± 3 | 0.020 |
| F10 | 70 | 67.8 ± 1.5 | 5.25 | 5.20 | 302 ± 3 | 0.019 |
| F11 | 10 | 9.9 ± 0.3 | 5.25 | 5.15 | 311 ± 1 | 0.020 |
| F12 | 100 | 92.6 ± 0.8 | 5.25 | 5.16 | 320 ± 4 | 0.021 |
| F13 | 100 | 97.4 ± 3.3 | 5.25 | 5.22 | 324 ± 2 | 0.022 |
| F14 | 100 | 96.6 ± 0.7 | 5.25 | 5.20 | 301 ± 2 | 0.021 |
| F15 | 100 | 95.4 ± 0.9 | 5.25 | 5.15 | 302 ± 3 | 0.021 |
| F17 | 100 | 101.2 ± 2.3 | 5.25 | 5.13 | 291 ± 4 | 0.018 |
| F18 | 100 | 100.3 ± 1.2 | 5.25 | 5.16 | 296 ± 1 | 0.043 |

### Example 3—Limited Analysis of Subvisible Particles at 6 Month Time Point

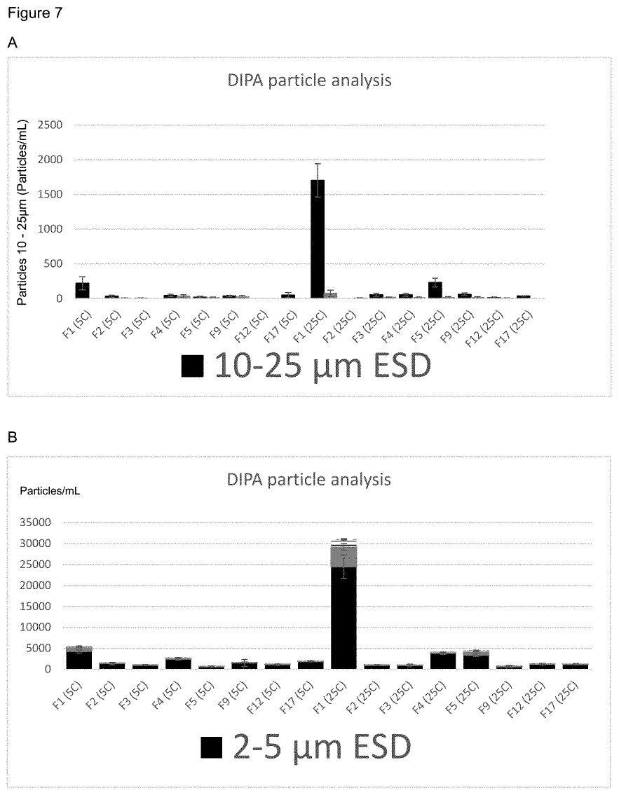

Low subvisible particle counts were measured for most of the formulations tested after storage for 3 months at both 5 and 25° C. in all particle-size ranges tested (see FIG. 7).

High sub visible particle counts were observed for F1 (at both 5° C. and 25° C.). This could be related to the process to generate the formulation. The high level of particles could be related to the pH adjustment, by spiking and not by dialysis.

### Example 4—Study of Aggregation Behavior (High Molecular Weight Species) by Size Exclusion Chromatography at T0 and Over Time

All formulations 1-18 (see Table 10) were analyzed by size exclusion chromatography for the quantitation of high molecular weight (soluble aggregates) species at T0, as described above.

Initial (T0) % HMW for all formulations are presented in Table 17. The aggregation rate constants, along with the corresponding standard error (SE), were determined for all formulations at 2-8, 25 and 40° C. by multiple linear regression as described before. The results are also summarized in Table 16.

Initial (T0) soluble aggregates profile for all formulations showed a variation in % HMW. This is attributed to differences in process when preparing different formulations. For example, some dialysates had to undergo up-concentration post dialysis while others did not. Overall, a relatively low % HMW was measured in all formulations at T0 (overall <1.5%). Interestingly, a systematic increase in aggregate (% HMW) levels was observed in formulations with increasing protein conc. (F2, F10, F11). Note that these formulations were prepared by simple dilution using the same buffer from the same stock dialysate solution.

Analysis of all formulations was discontinued after 3 months of storage at 40° C. since aggregation in all formulations reached levels sufficient to distinguish between performance of the formulations in terms of aggregation stability (best to worst performing). Analysis of F10, F11, F14 and F15 was also discontinued after 3 months at 2-8° C. and 25° C. Aggregation data collected to 3 months at 2-8° C. and 25° C. for F10 and F11 were sufficient to determine the impact of protein concentration on physical stability (by aggregation). F14 and F15, on the other hand, showed poor stability by this method and by other methods (to be discussed) sufficient to warrant their elimination. In general, % main species (monomer) was observed to decline with time along with a concurrent increase in aggregation (% HMW) and fragments (% LMW). Fragmentation however is not reported by this method but by a more reliable method for LMWs (CGE), as will be discussed later.

As expected, results in Table 16 show that the rate of aggregation increased with storage temperature. For every 10° C. increase in temperature, aggregation rate increased by 1.75 times on average up to 40° C. where more significant changes were observed, indicating possibly a different mechanism of aggregation of the molecule at this higher storage temperature as compared to lower temperature storage conditions.

To evaluate the effect of stabilizer type, the storage stability of F1-F7 was compared. Stabilizers used in the formulations were sugars/polyols, amino acids or mixtures of amino acids and sugars. Total stabilizer levels were fixed at 250 mM for these formulations. The results in Table 16 show that the Fc multimer molecule (CSL730) in the sucrose and trehalose based formulations (F2 and F3, respectively) had better storage stability as compared to all other formulations at all temperatures, as evidenced by slightly lower aggregation rates at 2-8° C. and 25° C. and significantly lower aggregation rates at 40° C. The sorbitol-based formulation showed superior stability to the amino acid based formulations (regardless of the presence or absence of sucrose with the amino acids) at 40° C., but comparable stability at lower temperatures for up to 6 months. Similarly, the proline-based formulations showed superior stability to the arginine-based formulations (regardless of the presence or absence of sucrose) at 40° C., but comparable stability at lower temperatures for up to 6 months.

To evaluate the effect of protein concentration at fixed stabilizer level, the storage stability of F2, F10 and F11 was compared for sucrose as a sugar type stabilizer. Protein concentration range was from 10-100 mg/mL. As expected, results in Table 16 show that the Fc multimer molecule (CSL730) exhibited superior stability at very low concentration (10 mg/mL) in F11 followed by 70 mg/mL (F10) followed by 100 mg/mL (F2). A plot of the aggregation rate constant as a function of protein concentration for all three formulations at all temperatures demonstrate a non linear increase in aggregation rate with protein concentration (results not shown). Note that at fixed stabilizer levels, the molar ratio of stabilizer to protein increases as protein concentration decreases (Table 10)—a factor that may contribute to the significant improvement in storage stability.

To evaluate the effect of stabilizer levels at fixed (high) protein concentration (100 mg/mL), the storage stability of F2, F8 and F9 was compared for sucrose as a sugar type stabilizer. Stabilizer levels ranged from 200-300 mM. Results in Table 16 show that sucrose at 225 mM (F8) was the least stable sucrose formulation as compared to 250 mM (F2) and 300 mM (F9) formulations at all storage temperatures. F9 showed incrementally better storage stability than F2 at all temperatures. Similarly, the Fc multimer molecule (CSL730) in proline at 200 mM (F17) was significantly less stable than in proline at 250 mM at all storage temperatures. Interestingly, the Fc multimer molecule (CSL730) was more stable in all sucrose formulations as compared to the proline formulations at 40° C. storage temperature.

To evaluate the effect of antioxidant (type and level) at a fixed stabilizer level, the storage stability of F2, F12, F13, F14 and F15 was compared. Methionine (at 10 and 20 mM) and reduced glutathione GSH (at 1 and 10 mM) were used as antioxidants. Results in Table 16 show that with methionine as an antioxidant, the Fc multimer molecule (CSL730) showed incrementally (small) improvements in the storage stability (physical) with increase in methionine levels at 40° C. However, there were no significant differences between the physical stability of the formulations with and without methionine. Formulations containing reduced GSH showed significantly greater instability at 40° C., as compared to the formulation without antioxidant (F2) and with methionine as an antioxidant (F12, F13). Smaller differences in storage stability were observed between the GSH-containing formulations, as compared to F2, F12 and F13 at 25° C., but with the same outcome (less stability with reduced GSH formulations). Nevertheless, reduced GSH formulations (F14, F15) were eliminated after 3 months of storage stability since stability data by other methods showed significant instability of F14 and F15 to warrant their elimination.

To evaluate the effect of surfactant (PS80) level at a fixed stabilizer level, the storage stability of F2 and F18 was compared. PS80 levels were evaluated at 0.02% w/v and 0.04% w/v. No significant differences were observed between the storage stability (aggregation) of both formulations at 2-8° C. and at 25° C. for up to 6 months. However, the Fc multimer molecule (CSL730) was more stable at a lower PS80 concentration (F2) at 40° C.

### Example 4—Study of Fragmentation Behavior (Low Molecular Weight Species) by Non Reducing Capillary Gel Electrophoresis (NR-cGE) at T0 and Over Time

All formulations (see Table 10) were analyzed by non-reducing cGE using Caliper (NR Caliper) for the quantitation of low molecular weight (LMW) species (i.e., fragmentation) at T0, as described above. Placebo samples were also run and no interference from placebo ingredients (i.e., formulation inactive ingredients) with protein peaks was observed in cGE chromatograms (data not shown).

Initial (T0) % LMW total species for all formulations were measured. F15 had the highest T0 level of fragments, an observation also confirmed using SEC (data not shown). The initial (total) % LMW species measured in all formulations, except for F15, was relatively low ranging between 2.1-2.5%.

Except for F10 and F11, fragmentation behavior of the formulations was monitored using the NR-cGE “Caliper” method over time at different temperatures for select formulations since it is a relatively higher throughput method. The fragmentation rate constants, along with the corresponding standard error (SE), were determined at different storage temperatures by multiple linear regression as described before. The results are also summarized in Table 17.

Analysis of all formulations was discontinued after 3 months of storage at 40° C. since fragmentation in all formulations reached levels sufficient to distinguish between performance of the formulations in terms of fragmentation stability (best to worst performing). Analysis of F14 and F15 was also discontinued after 3 months at 2-8° C. and 25° C. since they showed poor stability by this method and by other methods (aggregation, oxidation) sufficient to warrant their elimination. In general, as was observed with SEC analysis, % main species was observed to decline with time along with a concurrent increase in aggregation (% HMW) and fragments (% LMW). Aggregation, however, is not reported by this method since it has been reported by SEC, a more reliable method for soluble aggregates.

Small fluctuations in fragmentation (within assay error) occurred in all formulations studied at 2-8° C. with time, as evidenced by very small changes in % LMW after 3 and after 6 months.

As expected, results in Table 17 show that the rate of fragmentation increased with storage temperature.

To evaluate the effect of stabilizer type, the storage stability of F1-F7 was compared. Table 17 results show that the Fc multimer molecule (CSL730) exhibited more significant fragmentation potential in the presence of arginine (regardless of the presence or absence of sucrose: F5 and F6) at both 25° C. and 40° C. Fragmentation was comparable for all other formulations at 25° C. At 40° C., less fragmentation was observed with sucrose and sorbitol (F2 and F4, respectively) formulations as compared to proline and trehalose formulations (F1 and F3, respectively), evidenced by lower fragmentation rates at 40° C.

To evaluate the effect of stabilizer levels at fixed protein concentration (100 mg/mL), the storage stability of F2, F8 and F9 was compared for sucrose as a sugar type stabilizer. Stabilizer levels ranged from 200-300 mM. Results in Table 17 show no major differences in fragmentation stability at 25° C. as a function of sucrose level. Surprisingly, the formulation with sucrose at 250 mM (F2) showed better fragmentation stability than the corresponding formulations at 225 mM and 300 mM sucrose at 40° C. With proline as a stabilizer, the Fc multimer molecule (CSL730) in proline at 200 mM (F17) showed slightly better stability with regards to fragmentation as compared to the formulation with 250 mM proline at 25° C. and at 40° C.

To evaluate the effect of antioxidant (type and level) at a fixed stabilizer level, the storage stability of F2, F12, F13, F14 and F15 was compared. Results in Table 17 show that there were small differences in fragmentation storage stability of the Fc multimer molecule (CSL730) in the presence versus absence of methionine as an antioxidant at 40° C. Stability with regards to fragmentation was slightly better in the absence of methionine at 25° C. for reasons we cannot explain. More significant instability with respect to fragmentation was observed only after 3 months of storage at both 25° C. and 40° C. with formulations containing reduced GSH. Thus, reduced GSH formulations (F14, F15) were eliminated after 3 months of storage since stability data by this method and other methods showed sufficient instability of F14 and F15 to warrant their elimination.

To evaluate the effect of surfactant (PS80) level at a fixed stabilizer level, the storage stability of F2 (0.02% w/v PS80) and F18 (0.04% w/v PS80) was compared. No differences were observed between the storage stability (fragmentation) of both formulations at 25° C. for up to 6 months. However, the Fc multimer molecule (CSL730) was more stable at a lower PS80 concentration (F2) at 40° C.

### Example 5—Study of Acidic and Basic Species by Cation Exchange Chromatography (CEX) at T0 and Over Time

All formulations (see Table 10) were analyzed by CEX for the quantitation of % acidic species and basic species at T0, as described above. Placebo samples were also run by CEX and no interference from placebo ingredients with protein peaks was observed in CEX chromatograms (data not shown).

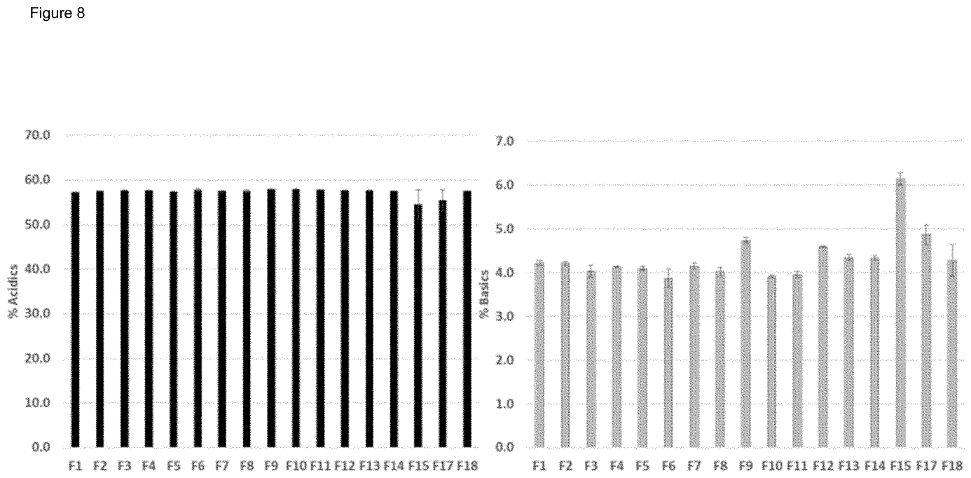

FIG. 8 compares the initial (T0) results for % acidic and basic species for all formulations. % acidic species at T0 for most formulations were comparable. Starting levels of % acidic species in F15 (with high reduced GSH levels) and F17 (with lower proline levels) was ˜2.5-3.5% lower as compared to the other formulations (also see Table 9). Similarly, % basic species at T0 for most formulations were comparable (˜4-5%). However, F15 had the highest starting levels of % basic species (6.1%) as compared to the other formulations.

% acidic species for all formulations was monitored using the CEX method over time at different temperatures. The rate constants for formation of acidic species, along with the corresponding standard error (SE), were determined at different storage temperatures by multiple linear regression as described before. The results are also summarized in Table 18.

Analysis of all formulations was discontinued after 2 months of storage at 40° C. since the levels of acidic species in all formulations were different and high enough to distinguish between performance of the formulations in terms of stability of acidic species (best to worst performing). Analysis of F10, F11, F14 and F15 was also discontinued after 3 months at 2-8° C. and 25° C. F14 and F15 showed poor stability by this method and by other methods sufficient to warrant their elimination. In general, % main species was observed to decline with time along with a concurrent increase in % basic species and a greater increase in acidic species.

F15 showed a significant rise in % acidic species after only 3 months of storage at 2-8° C. followed by F17. Small fluctuations in % acidic species (within assay error) were observed with the other formulations at the same temperature. For the formulations continued after the 3 month time point at 2-8° C., small fluctuations in % acidic species were observed in the same formulations. Among these formulations, F17 still showed higher levels of % acidic species after 6 months of storage at 2-8° C. (Table 18).

As expected, results in Table 18 show that the rate of formation of acidic species increased with storage temperature.

To evaluate the effect of stabilizer type, the storage stability of F1-F7 was compared. Table 18 results show that the Fc multimer molecule (CSL730) exhibited surprisingly better stability with regards to rate of formation of acidic species in the presence of arginine (F5 and F6) at both 25° C. and 40° C. (unlike fragmentation and aggregation stability trends).

To evaluate the effect of protein concentration at fixed stabilizer level, the storage stability of F2, F10 and F11 was compared for sucrose as a sugar type stabilizer. Results in Table 18 show that the Fc multimer molecule (CSL730) exhibited small differences in stability with regards to acidic species formation between the three formulations at different protein concentrations at both 25° C. and 40° C.

To evaluate the effect of stabilizer levels at fixed protein concentration (100 mg/mL), the storage stability of F2, F8 and F9 was compared for sucrose as a sugar type stabilizer. Stabilizer levels ranged from 200-300 mM. Results in Table 18 show that the Fc multimer molecule (CSL730) exhibited small differences in stability with regards to acidic species formation between the three formulations at different sucrose levels at both 25° C. and 40° C. In spite of starting higher levels of % acidic species with F17, the Fc multimer molecule (CSL730) in proline at 200 mM (F17) was comparable in stability with regards to acidic species formation in proline at 250 mM at both 25° C. and 40° C. Overall, the Fc multimer molecule (CSL730) stability with regards to acidic species formation in sucrose and proline formulations were comparable at both 25° C. and 40° C.

To evaluate the effect of antioxidant (type and level) at a fixed stabilizer level, the storage stability of F2, F12, F13, F14 and F15 was compared. Results in Table 18 show that there were small differences in storage stability with regards to acidic species formation of the Fc multimer molecule (CSL730) in the presence versus absence of methionine as an antioxidant at both 25° C. and 40° C. Similar to observations with aggregation and fragmentation stability, more instability with respect to acidic species formation was observed in formulations containing reduced GSH. A systematic increase in the rate constant for formation of acidic species with an increase in reduced GSH content up to 3 months at 25° C. and 2 months at 40° C. Thus, reduced GSH formulations (F14, F15) were eliminated after 3 months of storage stability since stability data by this method and other methods showed sufficient instability of F14 and F15 to warrant their elimination.

To evaluate the effect of surfactant (PS80) level at a fixed stabilizer level, the storage stability of F2 (0.02% w/v PS80) and F18 (0.04% w/v PS80) was compared. Small differences were observed between the storage stability (acidic species formation) of both formulations at 25° C. and at 40° C.

### Example 6—Study of Oxidation Behavior by Reverse Phase High Performance Liquid Chromatography (RP-HPLC) at T0 and Over Time

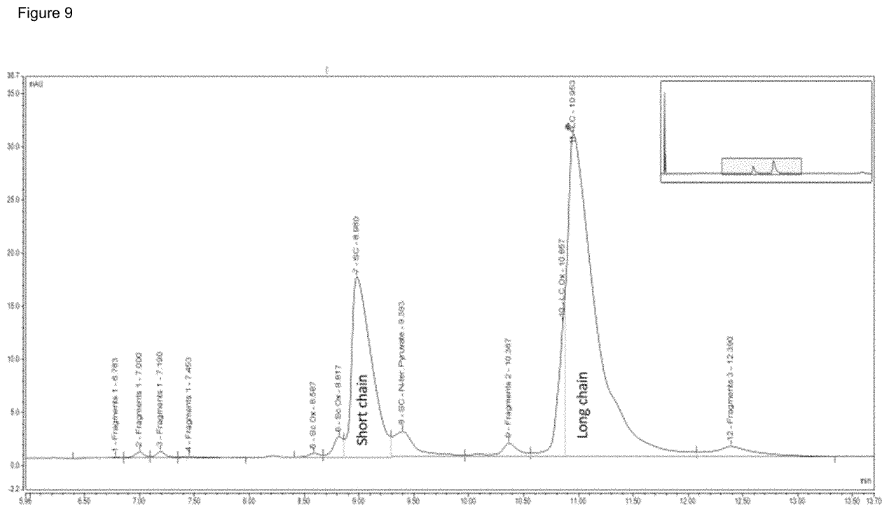

All formulations (see Table 10) were analyzed by RP-HPLC for the quantitation of total oxidation and % oxidation in long chain (LC) and short chain (SC) at T0, as described above. FIG. 9 shows representative chromatograms for the oxidation profile of the Fc multimer molecule (CSL730) in a sample formulation. FIG. 9 shows the elution of SC oxidized species followed by SC, then LC oxidized species followed by LC. The chromatogram profile also contains SC and LC fragments, as well as SC and LC N-tertiary pyruvate. Placebo samples were also run by the RP-HPLC method and no interference from placebo ingredients with protein peaks was observed in the chromatograms (data not shown). This was especially important to run for the formulations with reduced GSH.

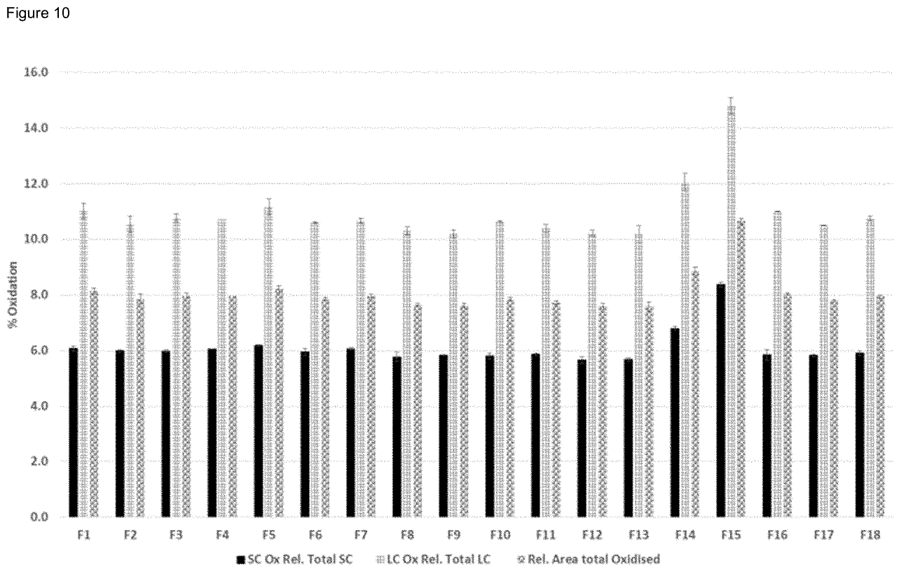

FIG. 10 compares the initial (T0) results for % SC oxidized species, % LC oxidized species and relative area (%) for total oxidation for all formulations. % acidic species at T0 for most formulations were comparable. Starting levels of % SC oxidized species, % LC oxidized species and relative area (%) for total oxidation for most formulations were comparable. On the other hand, higher levels of oxidation were observed by all measures in formulations with reduced GSH (F14 and F15) (also see Table 19). % oxidized species (SC and LC) and total % oxidation increased at T0 as reduced GSH levels increased.

% SC oxidized species, % LC oxidized species and relative area (%) for total oxidation for all formulations were monitored using the RP-HPLC method over time at different temperatures. The rate constants for total oxidation, along with the corresponding standard error (SE), were determined at different storage temperatures by multiple linear regression as described before. The results are also summarized in Table 19.

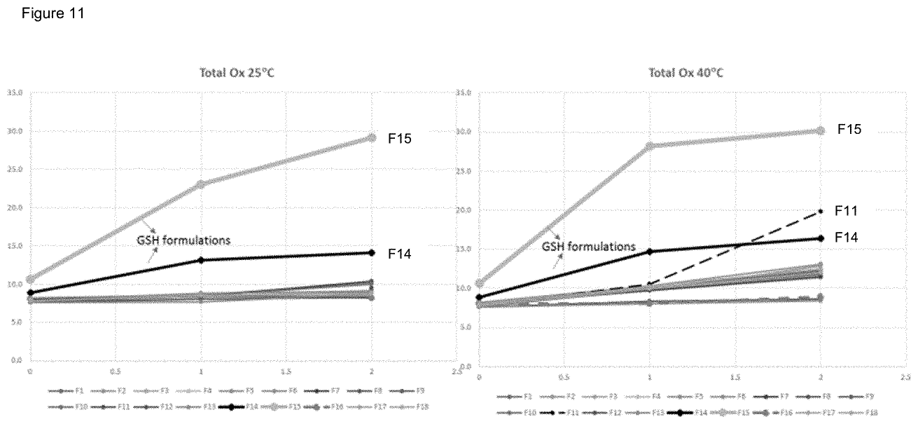

A few select formulations were analysed for up to 6 months at 2-8° C. and at 25° C. Oxidation analysis of some formulations was discontinued after only 2 months at 25° C. (based on their performance at 40° C.). Analysis of all formulations was discontinued after 2 months of storage at 40° C. since oxidation reached levels high enough to enable the identification and elimination of the worst performing formulations. In general, % unoxidized SC and unoxidized LC were observed to decline with time along with a concurrent increase in oxidized SC and % oxidized LC species. Overall, the total % oxidation in the different formulations increased at varying rates with time and temperature, as demonstrated in FIG. 11.

Table 19 shows that for formulations tested for oxidation after 6 months of storage at 2-8° C., they showed small increases in total % oxidation. However, the formulation containing arginine as the principle stabilizer (F5) showed the most oxidation after 6 months of storage—F14 and F15 not included at 2-8° C.

As expected, results in Table 19 show that the rate of oxidation increased with storage temperature. Peculiarly, the increase in the rate of oxidation at 25° C. and at 40° C. was very slight in presence of 10 mM reduced GSH (F15). This could possibly indicate that at high levels of GSH in the system studied (F15), oxidation becomes less temperature dependent and driven more by GSH concentration thus resulting in an alteration of oxidation kinetics.

To evaluate the effect of stabilizer type, the storage stability of F1-F7 was compared. Table 19 results show that the Fc multimer molecule (CSL730) exhibited better stability to oxidation in the presence of polyols/sugars at 25° C., as compared to amino acids. The outcome at 40° C. however, was not consistent with that at 25° C. Thus, no trend can be concluded from the results. Considering assay variability at the levels of oxidation measured, differences between the formulations would be considered small.

To evaluate the effect of protein concentration at fixed stabilizer level, the storage stability of F2, F10 and F11 was compared for sucrose as a sugar type stabilizer. Results in Table 19 show that the Fc multimer molecule (CSL730) exhibited more stability to oxidation as protein concentration increased at both 25° C. and 40° C. This appeared to be especially true at 40° C. when comparing the stability of the formulation at 10 mg/mL protein (F11) against the formulations at 100 mg/mL and 70 mg/mL protein (F2 and F10, respectively). F2 and F10 were 2.4-2.8 times more stable than F11 at 40° C.

To evaluate the effect of stabilizer levels at fixed protein concentration (100 mg/mL), the storage stability of F2, F8 and F9 was compared for sucrose as a sugar type stabilizer. Stabilizer levels ranged from 200-300 mM. Results in Table 19 show that the Fc multimer molecule (CSL730) exhibited slight differences in stability with regards to oxidation between the three formulations at different sucrose levels at both 25° C. and 40° C. The same outcome held true in the presence of proline as a stabilizer at a level of 200 mM and 250 mM at both 25° C. and 40° C.

To evaluate the effect of antioxidant (type and level) at a fixed stabilizer level, the storage stability of F2, F12, F13, F14 and F15 was compared. Results in Table 19 show small but systematic improvements in stability to oxidation with increasing levels of methionine at 25° C. (F12 and F13), as compared to its absence. A systematic but more significant improvement in stability to oxidation was observed at 40° C. with increasing levels of methionine. Formulations containing reduced GSH exhibited the poorest stability to oxidation, in contrast to formulations containing methionine (or even other formulations without antioxidants) (also see FIG. 11). A non-linear increase in the oxidation rate constant with an increase in reduced GSH content was observed for up to 2 months of storage at both 25° C. and 40° C. At both temperatures, just 1 mM GSH resulted in 4.7-9.3 times more oxidation that its counterpart formulations with methionine at both 25° C. and 40° C. Thus (as with other methods discussed previously), reduced GSH formulations (F14, F15) were eliminated based on only 2 months of oxidation storage stability.

To evaluate the effect of surfactant (PS80) level at a fixed stabilizer level, the storage stability of F2 (0.02% w/v PS80) and F18 (0.04% w/v PS80) was compared. No measurable differences were observed between the storage stability (oxidative) of both formulations at 40° C.

### Conclusions of Part 2

Formulation of a stable liquid DP of CSL730 at high concentration (≥100 mg/mL) is feasible
Stable to aggregation, acidic species formation, fragmentation and oxidation.
Factors contributing to stabilization of Fc multimer molecules, especially at high protein concentrations
Lower pH (5-5.8)
Use of disaccharides/polyols as stabilizers, and to a lesser extent amino acids
Optimum molar ratio for protein stability is a protein-to-sucrose ratio of 1:420. Stability also achieved with lower protein-to-sucrose ratios. Formulation robustness increases above that ratio.
Antioxidants may be required to prevent yellowing and/or side oxidation reactions that may occur due to other excipients during storage, or to protect against specific manufacturing conditions (e.g. light exposure, exposure to vaporized hydrogen peroxide residual from sterilization of filling machines in isolators) or unusual handling conditions during clinical administration.
Higher PS80 levels may not be needed for storage stability but may be needed to ensure stability during manufacturing and clinical handling/administration.
   Part 3 Stabilizer Screening Study at 120-160 mg/ml Protein Conc.

A follow up study to the excipient screening studies was performed to investigate the effect of stabilizer type (stabilizers of interest based on Part 2 were: sucrose, trehalose and proline) in the presence of 20 mM Histidine, 0.02% w/v polysorbate 80 and 10 mM methionine on liquid formulations comprising an Fc multimer molecule (CSL730) at high protein concentrations. Protein concentration was varied but molar ratio of protein to stabilizer was fixed at approximately 1:420. pH of all formulations was fixed at 5.25±0.1. Overall, CSL730 formulations with protein concentrations ranging from 120-160 mg/mL were prepared and investigated for protein stability. Formulations prepared for this study are summarized in Table 20.

TABLE 20
Summary of formulations prepared for the follow-up study at higher protein conc.

| | Target | Target | | | | | |
|---|---|---|---|---|---|---|---|
| | CSL730 | PS80 | | | Stabilizer | Antioxidant | |
| Formulation | conc. | conc. | Target | Stabilizer | Conc. | type and | Molar ratio |
| No. | (mg/mL) | (% w/v) | pH | type | (mM) | conc. (mM) | (protein:stabilizer) |
| F1 | 120 ± 5 | 0.02 | 5.25 ± 0.1 | Sucrose | 305 | 10 mM | 1:417 |
| | | | | | | Methionine | |
| F2 | 160 ± 5 | 0.02 | 5.25 ± 0.1 | Sucrose | 380 | 10 mM | 1:417 |
| | | | | | | Methionine | |
| F3 | 120 ± 5 | 0.02 | 5.25 ± 0.1 | Trehalose | 305 | 10 mM | 1:417 |
| | | | | | | Methionine | |
| F4 | 160 ± 5 | 0.02 | 5.25 ± 0.1 | Trehalose | 380 | 10 mM | 1:417 |
| | | | | | | Methionine | |
| F5 | 120 ± 5 | 0.02 | 5.25 ± 0.1 | Proline | 305 | 10 mM | 1:417 |
| | | | | | | Methionine | |
| F6 | 160 ± 5 | 0.02 | 5.25 ± 0.1 | Proline | 305 | 10 mM | 1:417 |
| | | | | | | Methionine | |

### Methods Used for Analyzing the Stability of the Formulations:

Stability indicating methods were used to analyze the formulations at the different time points namely: Size exclusion chromatography (SEC), Cation exchange chromatography (CEX), Capillary Gel Electrophoresis (CGE) (Caliper) and reverse phase HPLC (RP-HPLC) for oxidation. In addition, pH, visual appearance (for color, turbidity and visible particles) and subvisible particles (SVP) of the formulations were monitored at the different time points.

Methods used for the study and purpose of each method are summarized in Table 21. Storage conditions and analysis time points up to now are summarized in Table 22.

TABLE 21
Analytical methods used for stability studies
of formulations and their purpose

| Method | Purpose |
|---|---|
| Visual inspection | Visible particles, colour, |
| | opalescence, turbidity |
| Dynamic Imaging Particle Analysis | Sub-visible particle count |
| (DIPA) | |
| pH measurement | pH testing |
| Osmolality | Osmolality measurement |
| Polysorbate 80 (PS80) testing | Quantitation of PS80 |
| UV spectroscopy | Protein content (A280) |
| Size exclusion chromatography (SEC) | High molecular weight species |
| | (soluble aggregates) |
| Cation exchange chromatography | Acidic, basic and main species |
| (CEX) | |
| Capillary Gel Electrophoresis | Low molecular weight species |
| (CGE) (Caliper) | |

TABLE 22
A summary of storage conditions and analysis time points

| Storage condition | Time points | |
|---|---|---|
| 2-8° C. | 0, 1, 3, 6, 9, 12 | months |
| 25° C. | 0, 1, 2, 3, 6, 9, 12 | months |
| 35° C. | 0, 1, 2, 3, 6 | months |

The analytical methods were carried out as described in Part 1 and Part 2 above.

### Example 1—Visual Appearance at T0 and Over Time

All filled vials were examined by visual appearance after fill/finish (100% visual inspection) by two inspectors (Insp 1 and Insp 2, as described above) and at each time point and temperature. There was a general alignment between examiners in most cases.

All formulations possessed a brown yellow (BY) colour with slight opalescence due to high protein concentration. 0-1 visible particles were observed randomly in some formulations and placebos. A close examination, with knowledge about the protein, revealed that the visible particles were exogenous in nature (probably introduced during the fill/finish process). In spite of high viscosity, none of the formulations were found to be gelled.

Formulations 1 to 6 (see Table 20) were visually inspected for up to 12 months at different temperatures. The color of all formulations remained unchanged at all storage temperatures from T0 (BY). Clarity remained unchanged (slightly opalescent). Particle counts remained unchanged from T0 (0-1), regardless of temperature and time. None of the formulations gelled during the storage time period at any temperature.

### Example 2—Osmolality, pH, Protein Concentration and Polysorbate 80 Measurements at T0 and Over Time

Osmolality, pH, protein concentration and polysorbate 80 (PS80) concentration measurements on all formulations 1-6 (see Table 20) were conducted at the initial time point (T0). The results are summarized in Table 23.

The results in Table 23 show that protein concentration was within 120±5 mg/mL for F1, F3 and F5 and that protein concentration was within 160±5 mg/mL for F2, F4 and F6. pH measurements showed that the measured pH values were within ±0.1 pH units for most formulations in comparison to the target pH-values. A slightly higher deviation in pH was observed with F6 (within 0.17 units). Osmolality was generally within 382-414 mOsm/kg for formulations at 120 mg/ml whereas osmolality was generally higher (529-551 mOsm/kg) for formulations at 160 mg/ml. PS80 concentrations in all formulations were within a maximum of ±0.003% w/v of the PS80 target concentrations.

The pH and protein concentrations were monitored in formulations 1 to 6 (see Table 20) at 2-8, 25 and 35° C. for up to 12 months. Within experimental error, results showed minimum shifts (within a maximum of ±0.1 units) in pH at all temperatures for all formulations within the time frame measured. Also within experimental error, results showed small shifts in protein concentration with time.

TABLE 23
Measured protein concentration, pH, osmolality and polysorbate 80 (PS80)
concentration for all formulations at initial time point (T0)

| | Target | Measured | | | | |
|---|---|---|---|---|---|---|
| | protein | protein | | | Measured | PS80 |
| Form. | conc. | conc. | Target | Measured | Osmo. | conc. |
| Code | (mg/mL) | (mg/mL) | pH | pH | (mOsm/kg) | (% w/v) |
| F1 | 120 | 122 | 5.25 | 5.26 | 414 | 0.018 |
| F2 | 160 | 160 | 5.25 | 5.30 | 551 | 0.017 |
| F3 | 120 | 124 | 5.25 | 5.33 | 403 | TBD |
| F4 | 160 | 159 | 5.25 | 5.38 | 549 | 0.017 |
| F5 | 120 | 121 | 5.25 | 5.36 | 382 | 0.021 |
| F6 | 160 | 159 | 5.25 | 5.42 | 529 | 0.019 |

### Example 3—Study of Aggregation Behavior (High Molecular Weight Species) by Size Exclusion Chromatography at T0 and Over Time

All formulations 1-6 (see Table 20) were analyzed by size exclusion chromatography for the quantitation of high molecular weight (soluble aggregates) species at T0, as described above.

Initial (T0) % HMW for all formulations are presented in Table 24. The aggregation rate constants, along with the corresponding standard error (SE), were determined for all formulations at 2-8, 25 and 35° C. by multiple linear regression as described before. The results are also summarized in Table 24.

As shown in Table 24, initial (T0) soluble aggregates levels (% HMW) were comparable and low (within 0.8-1% HMW) in spite of high protein concentrations.

Analysis of all formulations was conducted up to 12 months of storage at 2-8° C., 25° C. and up to 6 months of storage at 35° C. As expected, results in Table 24 show that the rate of aggregation increased with storage temperature. Within the temperature ranges for the stability study, for every 10° C. increase in temperature, aggregation rate increased by up to 2 times.

The stability of the Fc multimer molecule (CSL730) at higher protein concentration (160 mg/ml) was on average 1.3 times less stable at 2-8° C., 25° C. and 35° C. as opposed to the same formulation at lower protein concentration (120 mg/ml).

For up to 3 months at fixed molar ratio of approximately 1:420, all stabilizers appeared to perform similarly with respect to stabilizing the Fc multimer molecule (CSL730) at all temperatures studied. This was also confirmed to be true up to 12 months.

TABLE 24a
Summary of SEC data for aggregation (% HMW) comparing
initial (T0) % HMW and aggregation rate constants k
(±SE) at 2-8° C., 25° C. and at 35° C. for 3 months

| | | Δ at 2-8° C. | | | |
|---|---|---|---|---|---|
| Form. | | after T = 3 | | | |
| Code | T0 | month | k_{2-8° C.} | k_{25° C.} | k_{40° C.} |
| F1 | 0.8 | 0.7 | 0.06 ± 0.01 | 0.15 ± 0.03 | 0.30 ± 0.05 |
| F2 | 0.9 | 0.8 | 0.06 ± 0.01 | 0.21 ± 0.04 | 0.40 ± 0.07 |
| F3 | 0.9 | 0.7 | 0.05 ± 0.01 | 0.16 ± 0.03 | 0.30 ± 0.05 |
| F4 | 1.0 | 0.9 | 0.06 ± 0.01 | 0.21 ± 0.04 | 0.40 ± 0.10 |
| F5 | 0.8 | 0.7 | 0.06 ± 0.01 | 0.15 ± 0.03 | 0.27 ± 0.05 |
| F6 | 0.9 | 0.9 | 0.07 ± 0.02 | 0.20 ± 0.04 | 0.36 ± 0.08 |

TABLE 24b
Summary of SEC data for aggregation (% HMW) comparing initial
(T0) % HMW and aggregation rate constants k (±SE) at 2-8°
C. and 25° C. for 12 months and at 35° C. for up to 6 months

| | | Δ at 2-8° C. | | | |
|---|---|---|---|---|---|
| Form. | | after T = 12 | | | |
| Code | T0 | month | k_{2-8° C.} | k_{25° C.} | K_{35° C.} |
| F1 | 0.8 | 1.86 | 0.15 ± 0.01 | 0.30 ± 0.04 | 0.98 ± 0.10 |
| F2 | 0.9 | 2.07 | 0.17 ± 0.02 | 0.40 ± 0.05 | 1.59 ± 0.28 |
| F3 | 0.9 | 1.79 | 0.14 ± 0.011 | 0.31 ± 0.04 | 0.97 ± 0.11 |
| F4 | 1.0 | 2.37 | 0.19 ± 0.01 | 0.39 ± 0.06 | (2.45 ± 0.14)* |
| F5 | 0.8 | 1.79 | 0.15 ± 0.01 | 0.28 ± 0.04 | 0.94 ± 0.10 |
| F6 | 0.9 | 2.40 | 0.19 ± 0.02 | 0.36 ± 0.05 | 1.14 ± 0.15 |

*Rate estimate not representative. Not enough sample available to perform time points >2 months

F1 to F6 all showed good stability for at least 12 months. At 4° C., all 6 formulations contained less than 3.5% HMW species after 12 months; at 25° C., all 6 formulations contained less than 6.5% HMW species.

### Example 4—Study of Fragmentation Behavior (Low Molecular Weight Species) by Non Reducing Capillary Gel Electrophoresis (NR-cGE) at T0 and Over Time

All formulations (see Table 20) were analyzed by non-reducing cGE using Caliper (NR Caliper) for the quantitation of low molecular weight (LMW) species (i.e., fragmentation) at T0, as described above.

Initial (T0) % LMW species for all formulations are presented in Table 25. As shown in Table 25, % LMWs at T0 for all formulations were comparable. The same outcome was observed with LMWs as measured by the SEC method at T0 (measured % LMWs in all formulations were in the range of 2.6-2.7%).

% LMW species for all formulations was monitored using the NR-cGE “Caliper” method over time at different temperatures, initially for 3 months, but now continued for 12 months. The rate constants for fragmentation, along with the corresponding standard error (SE), were determined at different storage temperatures by multiple linear regression as described before. The results are summarized in Table 25.

As expected, results in Table 25 show that the rate of fragmentation increased with storage temperature. Small fluctuations in fragmentation (within assay error) occurred in all formulations studied at 2-8° C. with time, as evidenced by small changes in % LMW for up to 12 months. However, for a 10° C. increase in storage temperature (from 25° C. to 35° C.), the rate of fragmentation increased on average by 3.6 to 4 times, regardless of protein concentration. A similar outcome was observed with rate of fragmentation as measured by the SEC method (results not shown). With the SEC method, the rate of fragmentation increased on average by 3.5 times for a 10° C. increase in storage temperature (from 25° C. to 35° C.).

At the same storage temperature, no differences were observed between the stability of the Fc multimer molecule (CSL730) at higher protein concentration (160 mg/ml) as opposed to the same formulation at lower protein concentration (120 mg/ml). That is, there were no significant differences between stability of the formulations at both 25° C. and 35° C. as a function of protein concentration. The same outcome was observed with LMWs as measured by the SEC method (results not shown).

For up to 12 months at a fixed molar ratio of approximately 1:420, all stabilizers appeared to perform similarly with respect to stabilizing the Fc multimer molecule (CSL730) against fragmentation at 2-8° C., 25° C. and at 35° C.

TABLE 25a
Summary of data from NR-cGE from “Caliper” method for fragmentation
(% LMW) comparing initial (T0) % LMW, changes in fragmentation
at 2-8° C. after 3 months, and fragmentation rate constants
k (±SE) at 25° C. and at 35° C. for 3 months of storage time.

| | | Δ at 2-8° C. | | |
|---|---|---|---|---|
| Form. | | after T = 3 | k_{25° C.} | K_{35° C.} |
| Code | T0 | months | (T = 3 months) | (T = 3 months) |
| F1 | 2.8 | 0.3 | 0.12 ± 0.01 | 0.36 ± 0.04 |
| F2 | 2.8 | 0.5 | 0.12 ± 0.01 | 0.40 ± 0.01 |
| F3 | 2.8 | 0.3 | 0.12 ± 0.04 | 0.40 ± 0.02 |
| F4 | 2.8 | 0.2 | 0.13 ± 0.04 | 0.40 ± 0.01 |
| F5 | 2.8 | 0.5 | 0.11 ± 0.04 | 0.42 ± 0.03 |
| F6 | 2.7 | 0.2 | 0.12 ± 0.04 | 0.41 ± 0.01 |

TABLE 25b
Summary of NR-cGE data from “Caliper” method
for fragmentation (% LMW) comparing initial (T0) %
LMW and fragmentation rate constants k (±SE) at
2-8° C. and 25° C. for 12 months and at 3 5° C. for up to 6 months

| | | Δ at 2-8° C. | | | |
|---|---|---|---|---|---|
| Form. | | after T = 12 | | | |
| Code | T0 | month | k_{2-8° C.} | k_{25° C.} | K_{35° C.} |
| F1 | 2.8 | 0.4 | ≤0.05 | 0.48 ± 0.03 | 1.74 ± 0.12 |
| F2 | 2.8 | 0.2 | ≤0.05 | 0.45 ± 0.04 | 1.60 ± 0.03 |
| F3 | 2.8 | 0.2 | ≤0.05 | 0.43 ± 0.03 | 1.74 ± 0.06 |
| F4 | 2.8 | 0.4 | ≤0.05 | 0.41 ± 0.03 | (2.40 ± 0.40)* |
| F5 | 2.8 | 0.2 | ≤0.05 | 0.44 ± 0.03 | 1.74 ± 0.05 |
| F6 | 2.7 | 0.3 | ≤0.05 | 0.46 ± 0.03 | 1.83 ± 0.06 |

*Rate estimate not representative. Not enough sample available to perform time points >2 month

### Example 5—Study of Acidic and Basic Species by Cation Exchange Chromatography (CEX) at T0 and Over Time

All formulations (see Table 20) were analyzed by CEX for the quantitation of % acidic species and basic species at T0, as described above.

Initial (T0) % acidic species for all formulations are presented in Table 26. As shown in Table 26, % acidic species at T0 for all formulations were comparable. Similarly, % basic species at T0 for all formulations were also comparable (3.5-3.7%).

% acidic species for all formulations was monitored using the CEX method over time at different temperatures (up to 12 months). The rate constants for formation of acidic species, along with the corresponding standard error (SE), were determined at different storage temperatures by multiple linear regression as described before. The results are summarized in Table 26.

As expected, results in Table 26 show that the rate of formation of acidic and basic species increased with storage temperature. For up to 12 months at 2-8° C., no changes were observed in acidic species formation for any of the formulations studied. However, for a 10° C. increase in storage temperature (from 25° C. to 35° C.), the rate of acidic species formation increased on average by 3.1-3.4 times.

Results in Table 26 show that the stability of the Fc multimer molecule (CSL730) at lower protein concentration (120 mg/ml) was slightly better at a storage temperature of 25° C. than the same formulation at higher protein concentration (160 mg/ml).

Results in Table 26 also show that at a fixed molar ratio of approximately 1:420, stability of Fc multimer molecule (CSL730) with regards to acidic species formation in the presence of sucrose, trehalose or proline at 25° C. was comparable. Results at 35° C. are less decisive due to fewer samples for analysis at that temperature.

TABLE 26
Summary of data from CEX for % acidic species comparing initial
(T0), changes in % acidic species at 2-8° C. after 3
months, and rate constants for formation of acidic species
k (±SE) at 25° C. and at 40° C. for 3 months of storage time

| | | Δ at 2-8° C. | | |
|---|---|---|---|---|
| Form. | T0 | after T = 12 | | |
| Code | (Acidic) | month | k_{25° C.} | K_{35° C.} |
| F1 | 56.9 | 0.1 | 1.37 ± 0.03 | 4.72 ± 0.46 |
| F2 | 56.8 | 0.0 | 1.33 ± 0.04 | (5.63 ± 0.31)* |
| F3 | 56.9 | 0.1 | 1.40 ± 0.03 | 4.57 ± 0.51 |
| F4 | 56.8 | 0.0 | 1.32 ± 0.05 | (8.65 ± 0.78)* |
| F5 | 56.9 | 0.4 | 1.54 ± 0.04 | 4.80 ± 0.58 |
| F6 | 56.9 | 0.0 | 1.44 ± 0.04 | 4.68 ± 0.55 |

*Rate estimates not representative. Not enough sample available to perform time points >2-3 months

No significant differences were observed among the 6 formulations for % basic species for the different time points and temperatures.

1. An Fc multimer composition comprising
a) an Fc multimer at a concentration between 60 mg/ml and 180 mg/ml,
b) a pH value between 4.8 and 6.0, and
c) a stabilizer at a concentration between 200 mM and 450 mM.

2. The Fc multimer composition of claim 1, comprising an Fc multimer at a concentration between 70 mg/ml and 110 mg/ml.

3. The Fc multimer composition of claim 1, comprising an Fc multimer at a concentration between 110 mg/ml and 180 mg/ml.

4. The Fc multimer composition of claim 1, wherein the composition comprises a buffer.

5. The Fc multimer composition of claim 4, wherein the pH value is between 5.0 and 5.5.

6. The Fc multimer composition of claim 4, wherein the buffer is an acetate buffer, a histidine buffer, or a citrate buffer.

7. The Fc multimer composition of claim 1, wherein the stabilizer is a polyol.

8. The Fc multimer composition of claim 1, wherein the molar ratio of Fc multimer to stabilizer is between 1:300 and 1:650.

9. The Fc multimer composition of claim 1, further comprising an antioxidant.

10. The Fc multimer composition of claim 1, further comprising a surfactant.

11. The Fc multimer composition of claim 1 in lyophilized form.

12. The Fc multimer composition of claim 1, wherein the Fc multimer is composed of 3 to 10 Fc portions.

13. The Fc multimer composition of claim 12, wherein the Fc multimer is composed of 3 Fc portions.

14. The Fc multimer composition of claim 13, wherein each Fc portion comprises 2 Fc polypeptides, which each comprise a hinge region, a CH2 constant domain, and a CH3 constant domain.

15. The Fc multimer composition of claim 14, wherein the Fc multimer comprises two polypeptides having the amino acid sequence of SEQ ID NO: 23 and two polypeptides having the amino acid sequence of SEQ ID NO: 24.

16. (canceled)

17. A method of treating an autoimmune disease or an inflammatory disease in a subject in need thereof, comprising administering to the subject a therapeutically effective amount of the Fc multimer composition of claim 1.

18. The Fc multimer composition of claim 7, wherein the polyol is a disaccharide.

19. The Fc multimer composition of claim 18, wherein the disaccharide is sucrose, trehalose, or sorbitol.

20. The Fc multimer composition of claim 9, wherein the antioxidant is methionine.

21. The Fc multimer composition of claim 10, wherein the surfactant is polysorbate 80.
